(19) United States
(12) Patent Application Publication
(10) Pub. No.: US 2016/0367198 A1
(43) Pub. Date: Dec. 22, 2016

(54) APPARATUS AND METHOD FOR DETECTING AND REMOVING ARTIFACTS IN OPTICALLY ACQUIRED BIOLOGICAL SIGNALS

(71) Applicants: Ki H. Chon, Worcester, MA (US); Jo Woon Chong, Southbridge, MA (US); Yitzhak Mendelson, Worcester, MA (US); S.M.A. Salehizadeh, Worcester, MA (US); Duy K. Dao, Malden, MA (US)

(72) Inventors: Ki H. Chon, Worcester, MA (US); Jo Woon Chong, Southbridge, MA (US); Yitzhak Mendelson, Worcester, MA (US); S.M.A. Salehizadeh, Worcester, MA (US); Duy K. Dao, Malden, MA (US)

(21) Appl. No.: 15/121,277

(22) PCT Filed: Feb. 26, 2015
(86) PCT No.: PCT/US2015/017746

§ 371 Date: Aug. 24, 2016

Related U.S. Application Data
(60) Provisional application No. 61/944,726, filed on Feb. 26, 2014.

(51) Int. Cl. A61B 5/00; A61B 5/024

(52) U.S. Cl. CPC A61B 5/7267; A61B 5/02416; A61B 5/7207

(57) ABSTRACT

Systems and methods that can distinguish clean from corrupted PPG signals under various types of motions and reconstruct the MNA contaminated data segments, such that biological parameters, e.g., heart rates and SpO2 values, can be accurately estimated, are disclosed.

### BACKGROUND

These teachings relate generally to an apparatus and a method for detecting and removing artifacts in optically acquired biological signals. More particularly, these teachings relate generally to an apparatus and a method for detecting and reconstructing motion and noise artifacts (MNA) in photoplethysmography (PPG) signals.

PPG is a non-invasive and low cost device to continuously monitor blood volume changes in peripheral tissues. PPG is a useful technique since it is widely used to monitor heart rate (HR), arterial oxygen saturation (SpO2), and can also be used to measure respiratory rates. However, MNA can distort PPG recordings, causing erroneous estimation of HR and SpO2. There are three distinct sources of MNA artifacts that can distort PPG recordings: (1) environmental, physiological, and experimental artifacts, which can be attributed to power interference surrounding the body; (2) correlated dynamics from other physiological signals; and (3) instrumental noise, respectively. MNA, which are comprised of all of the aforementioned noise sources, are difficult to filter since they do not have a predetermined frequency band and their spectrum often overlaps with that of the desired PPG signal.

MNA in PPG readings are caused by 1) the movement of venous blood as well as other non-pulsatile components along with pulsatile arterial blood and 2) variations in the optical coupling between the sensor and the skin. Various approaches to mitigate motion artifacts by improving sensor attachment have been proposed. However, these design improvements do not provide a significant reduction of motion artifacts. Algorithm-based MNA reduction methods are also proposed. These include time and frequency domain filtering, power spectrum analysis, and blind source separation techniques. However, these have high computational complexity and more importantly, they operate even on clean PPG portions where MNA reduction is not needed. Hence, accurate MNA detection, which identifies clean PPG recordings from corrupted portions, is essential for the subsequent MNA reduction algorithm so that it does not distort the non-corrupted data segments. Moreover, more computationally efficient MNA algorithms can be designed since they can be tailored only to the MNA contaminated data segments.

MNA detection methods are mostly based on a signal quality index (SQI) which quantifies the severity of the artifacts. Some approaches quantify SQI using waveform morphology or filtered output, while others derive SQI with the help of additional hardware such as accelerometer and electrocardiogram sensing. Statistical measures, such as skewness, kurtosis, Shannon entropy, and Renyi's entropy, have been shown to be helpful in determining a SQI. However, these techniques require manual threshold settings for each parameter to classify if the PPG signal is clean or corrupted. Although a support vector machine (SVM)-based classification method addresses the need of threshold setting, this approach considers limited and controlled types of motions.

On the other hand, arterial oxygen saturation reflects the relative amount of oxyhemoglobin in the blood. The most common method to measure it is based on pulse oximetry, whereby oxidized hemoglobin and reduced hemoglobin have significantly different optical spectra. Specifically, at a wavelength of about 660 nm, and a second wavelength between 805 and 960, there is a large difference in light absorbance between reduced and oxidized hemoglobin. A measurement of the percent oxygen saturation of blood is defined as the ratio of oxyhemoglobin to the total concentration of hemoglobin present in the blood. Pulse oximetry assumes that the attenuation of light is due to both the blood and bloodless tissue. Fluctuations of the PPG signal are caused by changes in arterial blood volume associated with each heartbeat, where the magnitude of the fluctuations depends on the amount of blood rushing into the peripheral vascular bed, the optical absorption of the blood, skin, and tissue, and the wavelength used to illuminate the blood.

The pulse oximeter signal contains not only the blood oxygen saturation and heart rate data, but also other vital physiological information. The fluctuations of PPG signals contain the influences of arterial, venous, autonomic and respiratory systems on the peripheral circulation. In the current environment where health care costs are ever increasing, a single sensor that has multiple functions is very attractive from a financial perspective. Moreover, utilizing a pulse oximeter as a multi-purpose vital sign monitor has clinical appeal, since it is familiar to the clinician and comfortable for the patient. Knowledge of respiratory rate and heart rate patterns can provide more useful clinical information in many situations in which pulse oximeter is the sole monitor available.

Although there are many promising and attractive features of using pulse oximeters for vital sign monitoring, currently they are used on stationary patients. This is mainly because MNA result in unreliable heart rate and SpO2 estimation. Clinicians have cited motion artifacts in pulse oximetry as the most common cause of false alarms, loss of signal, and inaccurate readings.

In practice, MNA are difficult to remove because they do not have a predefined narrow frequency band and their spectrum often overlaps that of the desired signal. Consequently, development of algorithms capable of reconstructing the corrupted signal and removing artifacts is challenging.

There are a number of general techniques used for artifact detection and removal. One of the methods used to remove motion artifacts is adaptive filtering. An adaptive filter is easy to implement and it also can be used in real-time applications, though the requirement of additional sensors to provide reference inputs is the major drawback of such methods.

There are many MNA reduction techniques based on the concept of blind source separation (BSS). BSS is attractive and has garnered significant interest since this approach does not require a reference signal. The aim of the BSS is to estimate a set of uncorrupted signals from a set of mixed signals which is assumed to contain both the clean and MNA sources. Some of the popular BSS techniques are independent component analysis (ICA), canonical correlation analysis (CCA), principle component analysis (PCA), and singular spectrum analysis (SSA).

In ICA, the recorded signals are decomposed into their independent components or sources. CCA uses the second order statistics (SOS) to generate components derived from their uncorrelated nature. PCA is another noise reduction technique which aims to separate the clean signal dynamics from the MNA data. A multi-scale PCA has also been proposed to account for time-varying dynamics of the signal and motion artifacts from PPG recordings.

A promising approach that can be applied to signal reconstruction is the singular spectrum analysis (SSA). The SSA is a model-free BSS technique, which decomposes the data into a number of components which may include trends, oscillatory components, and noise (see, for example, B. S. Kim and S. K. Yoo, “Motion artifact reduction in photoplethysmography using independent component analysis,” Biomedical Engineering, IEEE Transactions on, vol. 53, pp. 566-568, 2006, which is incorporated herein by reference in its entirety for all purposes.) The main advantage of SSA over ICA is that SSA does not require user input to choose the appropriate components for reconstruction and MNA removal. Comparing PCA to SSA, SSA can be applied in cases where the number of signal components is more than the rank of the PCA covariance matrix. Applications of the SSA include extraction of the amplitude and low frequency artifacts from single channel EEG recordings, and removing heart sound dynamics from respiratory signals.

Accordingly, there is a need to develop a new apparatus and a new method to distinguish clean from corrupted PPG signals under various types of motions. There is also a need to develop a new apparatus and a new method to remove MNA from corrupted PPG signals and to reconstruct PPG signals from the corrupted PPG signals.

### BRIEF SUMMARY

In view of the foregoing, these teachings provide systems and methods that can distinguish clean from corrupted PPG signals under various types of motions and reconstruct the MNA contaminated data segments, such that biological parameters, e.g., heart rates and SpO2 values, can be accurately estimated.

In one embodiment, the system of these teachings includes one or more processors and one or more computer usable media having computer readable code embodied therein, the computer readable code causing the one or more processors to execute the method of these teachings.

In another embodiment, the method of these teachings includes a method for determining MNA are present in a segment of PPG data by determining a plurality of time domain features for each segment from a plurality of test segments of the PPG data, the plurality of test segments including segments without motion and noise artifacts and other segments with motion and noise artifacts, the plurality of time domain features for said each segment from the plurality of test segments constituting a training set, using the training set to train a SVM, training resulting in a trained SVM, determining the plurality of time domain features for the segment, and using the trained SVM to determine whether motion and noise artifacts are present in the segment.

In yet another embodiment, the method of these teachings includes a method for removal of MNA present in a segment of PPG data, by the steps of: (a) for each one segment from a segment of PPG data in which presence of motion and noise artifacts has been previously detected, referred to as a corrupted segment, and a most prior adjacent segment of PPG data in which motion and noise artifacts are not detected, referred to as a clean segment, performing the following: (a1) assemble a data transition matrix, each row of the data transition matrix being a vector of a predetermined length, a number of vectors being equal to a number of samples in a segment for which the data transition matrix is assembled minus the predetermined length and plus one; a starting value of each vector being displaced by one sample from a previous vector, resulting in the data transition matrix having a number of columns equal to the predetermined length and a number of rows equal to the number of vectors; (a2) obtain eigenvectors and eigenvalues for the data transition matrix, resulting in eigenvectors and eigenvalues for the corrupted segment and eigenvectors and eigenvalues for the clean segment; (b) sorting the eigenvalues for the corrupted segment from largest to smallest; and sorting the eigenvalues for the clean segment from largest to smallest; (c) retaining only a top predetermined percentage of the eigenvalues for the corrupted segment and the eigenvalues for the clean segment; (d) replacing the eigenvalues for the corrupted segment with the eigenvalues for the clean segment, where only the top predetermined percentage of the eigenvalues and corresponding eigenvectors have been retained; (e) retaining only eigenvectors for the corrupted segment and eigenvectors for the clean segment that have data in a predetermined frequency range; (f) discarding eigenvectors for the corrupted segment that have different frequencies from the eigenvectors for the clean segment; (g) obtaining the data transition matrix for the corrupted segment from the eigenvalues and eigenvectors of the corrupted segment and the data transition matrix for the clean segment from the eigenvalues and eigenvectors of the clean segment; (h) repeating steps (a2) to (g) until a predetermined convergence criterion is satisfied; and (i) reconstructing, after the predetermined convergence criterion is satisfied, the corrupted segment from the data transition matrix for the corrupted segment using replaced eigenvalues and retained eigenvectors.

In still another embodiment, the system of these teachings includes a system for determining whether MNA are present in a segment of PPG data, having one or more processors and non-transitory computer usable media having computer readable code embodied therein, the computer readable code, when executed by the one or more processors, causes the one or more processors to: determine a plurality of time domain features for each segment from a plurality of test segments of the PPG data, the plurality of test segments including segments without motion and noise artifacts and other segments with motion and noise artifacts, the plurality of time domain features for said each segment from the plurality of test segments constituting a training set; use the training set to train a SVM, training resulting in a trained SVM; determine the plurality of time domain features for the segment; and use the trained SVM to determine whether motion and noise artifacts are present in the segment.

In yet another embodiment, the system of these teachings includes a system for removal of MNA present in a segment of PPG data, having one or more processors and non-transitory computer usable media, having computer readable code embodied therein, the computer readable code, when executed by the one or more processors, causes the one or more processors to: (a) for each one segment from a segment of PPG data in which presence of motion and noise artifacts has been previously detected, referred to as a corrupted segment, and a most prior adjacent segment of PPG data in which motion and noise artifacts are not detected, referred to as a clean segment, performing the following: (a1) assemble a data transition matrix, each row of the data transition matrix being a vector of a predetermined length, a number of vectors being equal to a number of samples in a segment for which the data transition matrix is assembled minus the predetermined length and plus one; a starting value of each vector being displaced by one sample from a previous vector, resulting in the data transition matrix having a number of columns equal to the predetermined length and a number of rows equal to the number of vectors; (a2) obtain eigenvectors and eigenvalues for the data transition matrix, resulting in eigenvectors and eigenvalues for the corrupted segment and eigenvectors and eigenvalues for the clean segment; (b) sorting the eigenvalues for the corrupted segment from largest to smallest; and sorting the eigenvalues for the clean segment from largest to smallest; (c) retaining only a top predetermined percentage of the eigenvalues for the corrupted segment and the eigenvalues for the clean segment; (d) replacing the eigenvalues for the corrupted segment with the eigenvalues for the clean segment, where only the top predetermined percentage of the eigenvalues and corresponding eigenvectors have been retained; (e) retaining only eigenvectors for the corrupted segment and eigenvectors for the clean segment that have data in a predetermined frequency range; (f) discarding eigenvectors for the corrupted segment that have different frequencies from the eigenvectors for the clean segment; (g) obtaining the data transition matrix for the corrupted segment from the eigenvalues and eigenvectors of the corrupted segment and the data transition matrix for the clean segment from the eigenvalues and eigenvectors of the clean segment; (h) repeating steps (a2) to (g) until a predetermined convergence criterion is satisfied; and (i) reconstructing, after the predetermined convergence criterion is satisfied, the corrupted segment from the data transition matrix for the corrupted segment using replaced eigenvalues and retained eigenvectors.

### BRIEF DESCRIPTION OF THE DRAWINGS

For a better understanding of the present teachings, together with other and further objects thereof, reference is made to the accompanying drawings and detailed description and its scope will be pointed out in the appended claims.

FIG. 1. A representative clean forehead-PPG signal recorded during voluntary motion artifact conducted in a laboratory setting (1st row). The mixed (up-down and left-right) movement of the forehead to which the PPG probe is attached for predetermined time interval induced 10% to 50% noise (2nd-6th row) within a 60s PPG segment.

FIG. 2. Training phase of the disclosed SVM-based motion detection algorithm. Four time-domain features corresponding to (1) standard deviation of peak-to-peak intervals (2) standard deviation of peak-to-peak amplitudes (3) standard deviation of systolic and diastolic interval ratio, and (4) mean standard deviation of pulse shape, are candidate input variables to the SVM.

FIG. 3. Test phase of the disclosed SVM-based motion detection algorithm. The hidden layers correspond to kernel function of the SVM. The function between hidden layer and output layer is a linear operator.

FIG. 4. Enhancement of MNA detection by diversity. Neighbor segments are the segments surrounding a target segment within ±2 seconds. Decisions on the target segment are based on a majority vote from the decisions of neighbor segments as well as the one of the target segment (red).

FIG. 5A-F. A sample forehead recorded PPG signal (a) along with the (b) standard deviation of P-P intervals (c) standard deviation of P-P amplitudes (d) standard deviation of systolic and diastolic time ratio, and (e) mean standard deviation of pulse shape, computed for each segment. The normalized sampled corrupt and clean PPGs for mean standard deviation of pulse shape is given in (f).

FIG. 6A-B. Trained SVM classification with a sample training finger recorded PPG signal is given with (a)-(b) pairs of two parameters. The SVM decision and margin boundaries are marked by black and green lines, respectively.

FIG. 7A-B. Validation: pairs of parameters for clean and corrupted PPG signals.

FIG. 8. A representative PPG signal with detected peaks (red) (a) along with the (b) standard deviation of P-P intervals (c) standard deviation of P-P amplitudes (d) mean standard deviation of pulse shape and (e) standard deviation of systolic and diastolic time ratio, computed for each segment

FIG. 9. Detection Probability of Corruption by additive white Gaussian noise (AWGN) for varying SNR from −20 to 0 dB. 50 AWGN realizations for each SNR level are separately added to a non-MNA corrupted PPG. Each realization is tested by the disclosed MNA detection algorithm to compute the detection probability of corruption

FIG. 10A-C. Classification performance comparison between our SVM algorithm, Hjorth (H1, H2), Kurtorsis and Shanon Entropy (K, SE) parameters. (a) Accuracy; (b) Sensitivity; (c) Specificity. The central mark on each box corresponds to the median; the edges of the box correspond to the 25th and 75th percentiles, the whiskers extend to the most extreme data points not considered outliers, and outliers are plotted individually. (*) indicate the mean is significantly different (p<0.05 at 95% CI) between SVM and other methods used for comparison

FIG. 11A-B. Comparison of mean errors and detection error fraction between original signal (labeled “None”) and artifact removed signal from five detection methods (SVM, H1, H2, K, and SE). (a) HR error; (b) SpO2 error

FIG. 12A-C. Mean error comparison between our SVM algorithm, Hjorth (H1, H2), Kurtorsis and Shanon Entropy (K, SE) parameters. (a) heart rate; (b) SpO2; (c) detection error. The central mark on each box corresponds to the median; the edges of the box correspond to the 25th and 75th percentiles, the whiskers extend to the most extreme data points not considered outliers, and outliers are plotted individually. (*) indicate the mean is significantly different (p<0.05 at 95% CI) between SVM and other methods used for comparison. The x-axis labeled “None” in all panels refers to the mean errors when compared to the reference signals without removing the MNA detected segments as identified by any of the five computational methods

FIG. 13. Typical infrared PPG signal; (a) clean, (b) corrupted with motion artifacts.

FIG. 14A-B. The first 12 eigenvector components of the PPG signal for: (a) Clean Infrared PPG, (b) Corrupted Infrared PPG.

FIG. 15A-C. Iterative reconstruction of a corrupted eigenvector with frequency of 0.967 Hz. Black font signals (top panels) represent the clean component with frequency of 0.967 Hz; Blue font signals (2nd rows) indicate the corrupted component with the same frequency; Pink font signals are related to iterative evolution of corrupted component to a clean oscillatory signal. (a) Reconstruction of 4th corrupted eigenvector compared to the corresponding clean component. The final pattern after 4 iterations resembles the black font clean component in the top panel. This component is chosen among the components with the same frequency, since it shows the most similarity to the black font clean component. (b) Reconstruction of 9th corrupted eigenvector compared to the corresponding clean component. (c) Reconstruction of 22nd corrupted eigenvector compared to the corresponding clean component

FIG. 16A1-B7. (Left) HR estimated from reconstructed PPG for different additive white noise levels; (Right) SpO2 estimated from reconstructed PPG for different levels of additive white noise

FIG. 17A1-B7. (Left) HR estimated from reconstructed PPG for different additive colored noise levels; (Right) SpO2 estimated from reconstructed PPG for different levels of additive colored noise.

FIG. 18A-D. (a) HR estimated from IMAR-reconstructed PPG compared to reference and corrupted PPG; (b) HR estimated from ICA-reconstructed PPG compared to reference and corrupted PPG; (c) SpO2 estimated from IMAR-reconstructed PPG compared to reference and corrupted PPG; (d) SpO2 estimated from ICA-reconstructed PPG compared to reference and corrupted PPG.

FIG. 19 is a schematic block diagram representation of one embodiment of the system of these teachings.

### DETAILED DESCRIPTION

The following detailed description presents the currently contemplated modes of carrying out the invention. The description is not to be taken in a limiting sense, but is made merely for the purpose of illustrating the general principles of the invention, since the scope of the invention is best defined by the appended claims.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.”

Motion and Noise Artifacts Detection

In these teachings, an accurate and comprehensive MNA detection algorithm is provided, which detects MNA in PPG under various types of motion. First, time-domain parameters are introduced to quantify MNA in the recorded PPG signal. Then, the statistical measures of the time-domain parameters are considered as input variables for a machine learning-based MNA detection algorithm. The MNA detection algorithm may be self-trained by the SVM with clean and corrupted PPG data sets, and then the trained SVM can be used to test the unknown PPG data. The efficacy of the MNA detection algorithm is tested on PPG data sets recorded from the finger and forehead pulse oximeters in simulations, laboratory-controlled and walking/stair-climbing experiments, respectively.

Experimental Protocol and Preprocessing

In order to further elucidate the teachings presented hereinbelow, data for exemplary embodiments was collected. PPG signals can be obtained from custom reflectance-mode prototype pulse oximeters. PPG data with laboratory-controlled head and finger movement, daily-activity movement, or simulated movement are collected respectively from healthy subjects recruited from the student community of Worcester Polytechnic Institute (WPI). This study is approved by WPI's IRB and all subjects are given informed consent prior to data recording.

In laboratory-controlled head movement data, motion artifacts are induced by head movements for specific time intervals in both horizontal and vertical directions. In one example, eleven healthy volunteers are asked to wear a forehead reflectance pulse oximeter along with a reference Masimo Radical (Masimo SET®) finger type transmittance pulse oximeter. After baseline recording for 5 minutes without any movement, subjects are instructed to introduce motion artifacts for specific time intervals varying from 10 to 50% within a 1 minute segment. For example, if a subject is instructed to perform left-right movements for 6 seconds, a 1 minute segment of data would contain 10% noise. The right middle finger with the sensor attached to the Masimo pulse oximeter is kept stationary. HR and SpO2 signals are acquired by the Masimo pulse oximeter at 80 Hz and 1 Hz, respectively, and are acquired synchronously with the PPG signals recorded from the forehead sensor.

In laboratory-controlled finger movement data, motion artifacts are induced by left-right movements of the index finger. In one example, nine healthy volunteers are asked to sit and wear two reflection type PPG pulse oximeters (TSD200) on their index and middle fingers, respectively. After baseline recording for 5 minutes without any movement to acquire clean data, motion artifacts are induced by left-right movements of the index finger while the middle finger is kept stationary as a reference. Similar to the head movement data, motion is induced at specific time intervals corresponding to 10-50% duration in a 1 minute segment. Such controlled movement is repeated five times per subject. The pulse oximeters are connected to a biopotential amplifier (PPG100) having a gain of 100 and cut-off frequencies of 0.05-10 Hz. The MP1000 (BIOPAC Systems Inc., CA, USA) is used to acquire finger PPG signals at 100 Hz. The daily-activity movement PPG data are recorded while subjects are walking straight or climbing stairs for 45 min. The nine subjects are asked to walk or climb stairs after wearing a forehead reflectance pulse oximeter along with a Holter electrocardiogram (ECG) monitor (Rozinn RZ153+) at 180 Hz and a Masimo Rad-57 pulse oximeter at 0.5 Hz. The reference ECG is obtained from the Holter ECG monitor while HR and SpO2 readings are measured from the Masimo pulse oximeter connected to the subject's right index finger, which is held against the chest to minimize motion artifacts. Finally, the simulation movement PPG data are generated by the addition of white noise to the clean PPG data.

PPG data are preprocessed by a 6th order infinite impulse response (IIR) band pass filter with cut-off frequencies of 0.5 Hz and 12 Hz. Zero-phase forward and reverse filtering is applied to account for the non-linear phase of the IIR filter. After these preprocessing, the following parameters for classifying clean and corruption are derived.

In one embodiment, the method of these teachings includes a method for determining whether MNA are present in a segment of PPG data by determining a plurality of time domain features for each segment from a plurality of test segments of the PPG data, the plurality of test segments including segments without motion and noise artifacts and other segments with motion and noise artifacts, the plurality of time domain features for said each segment from the plurality of test segments constituting a training set, using the training set to train a SVM, training resulting in a trained SVM, determining the plurality of time domain features for the segment, and using the trained SVM to determine whether motion and noise artifacts are present in the segment. The method also includes band pass before determining the plurality of time domain features, each segment from the plurality of test segments. The method still further includes determining whether motion and noise artifacts are present in segments neighboring the segment, referred to as neighboring segments, neighboring segments being segments surrounding the segment within a predetermined time interval. Finally, the method includes applying a majority vote algorithm to determinations of whether motion and noise artifacts are present in the segment and the neighboring segments. The time domain features include at least one of standard deviation of peak to peak interval within a segment, standard deviation of peak to peak amplitude within a segment, standard deviation of systolic and diastolic ratio within a segment, and mean standard deviation of pulse shape within an interval.

Parameters from PPG Signals

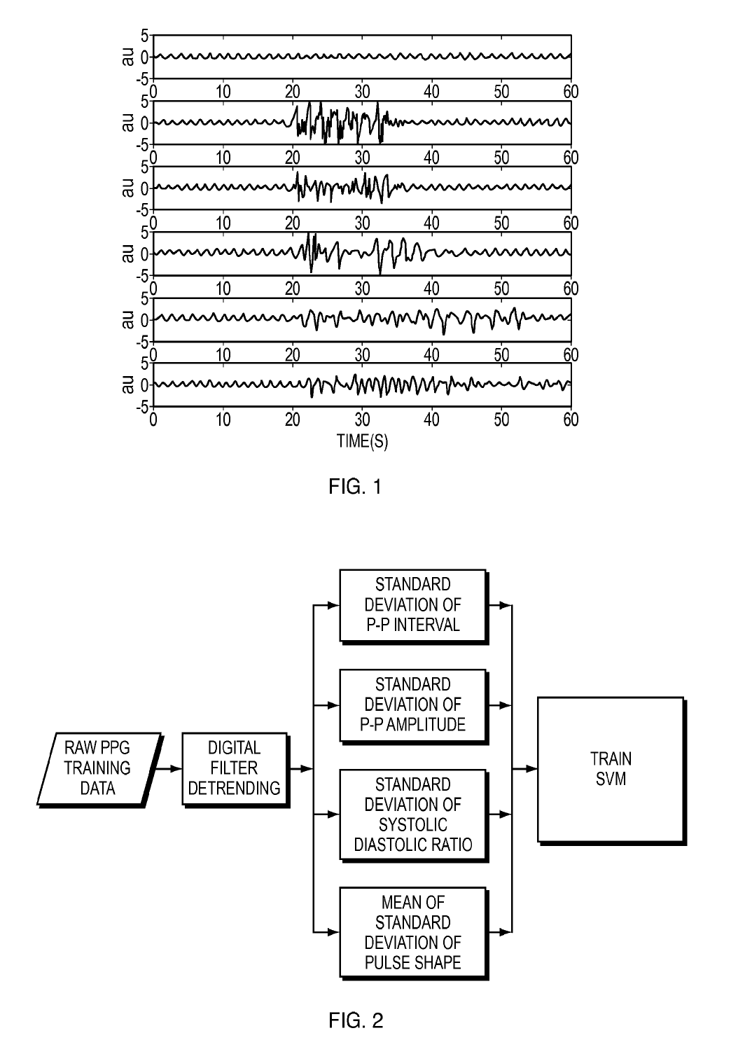

The following four parameters are selected since they represent the variability present in corrupted PPG signals as shown in FIG. 1.

1) Standard deviation of peak-to-peak interval (STD_{HR}):

The STD_{HR,n} of the n^{th} segment is defined by:

$${STD}_{HR , n} = \frac{1}{N - 1} {\sum }_{i = 1}^{N} ( {D}_{n , i} - \overline{{D}_{n}} ) \qquad ( 1 )$$

where D_{n,i} is peak-to-peak interval at the i^{th} pulse of the n^{th} segment and D̅ₙ̅ is mean peak-to-peak interval of the n^{th} segment. The D_{n,j} is calculated by the difference T_{peak,n,l}−T_{peak,n,l-1} between two successive peak times.

2) Standard deviation of peak-to-peak amplitude (STD_{AMP}): The STD_{AMP,n} of the n^{th} segment is defined by:

$${STD}_{AMP , n} = \frac{1}{N - 1} {\sum }_{i = 1}^{N} ( {A}_{n , i} - \overline{{A}_{n}} ) \qquad ( 2 )$$

where A_{n,i} is peak amplitude at the i^{th} pulse of the n^{th} segment and A̅ₙ̅ is mean peak-to-peak interval of the n^{th} segment. The A_{n,i} is defined by the difference between the i^{th} peak and the forthcoming (i+1)^{th} trough amplitudes.

3) Standard deviation of systolic and diastolic ratio (STD_{SD}): The STD_{SD,n} of the n^{th} segment is defined by:

$${STD}_{SD , n} = \frac{1}{N - 1} {\sum }_{i = 1}^{N} ( {R}_{SD , n , i} - \overline{{R}_{SD , n}} ) \qquad ( 3 )$$

where R_{SD,n,i} is systolic and diastolic time interval ratio at the i^{th} pulse of the n^{th} segment and R̅_̅{̅S̅D̅,̅n̅}̅ is the mean systolic and diastolic time interval ratio of the n^{th} segment. The R_{SD,n,i} is calculated by

R_{SD,n,i}=(T_{trough,n-1,i}−T_{peak,n,i})/(T_{peak,n,i}−T_{trough,n-1,i}) (4)

where T_{trough,n,i} denotes the trough (or lowest point) at the i^{th} pulse of the n^{th} segment.

4) Mean-standard deviation of pulse shape (STD_{WAV}): To derive pulse shape, we take Nₛₐₘₚ sample points of a pulse. The STD_{WAV,n} of the n^{th} segment is derived by taking average of the standard deviation at each sample point as follows:

STD_{WAV,n}=E[STD_{WAV,n,m}] (5)

where STD_{WAV,n,m} is calculated by:

$${STD}_{WAV , n , m} = \frac{1}{N - 1} {\sum }_{i = 1}^{N} ( {q}_{n , i} ( m ) - \overline{{q}_{n} ( m )} ) \qquad ( 6 )$$

where q_{n,i}(m) is the m^{th} pulse sample at the i^{th} pulse of the n^{th} segment and q̅ₙ̅(̅m̅)̅ is the mean at the m^{th} pulse sample of the n^{th} segment.

Classification by Support Vector Machine (SVM)

SVM can be applied to build a decision boundary classifying motion corruption from clean PPG signals. SVM is widely used in classification and regression due to its accuracy and robustness to noise (see, for example, C.-W. Hsu, C.-C. Chang, and C.-J. Lin, “A Practical Guide to Support Vector Classification,” Department of Computer Science, National Taiwan University 2003, a copy of which is incorporated by reference here in its entirety and for all purposes). The SVM includes training and test phases described further below.

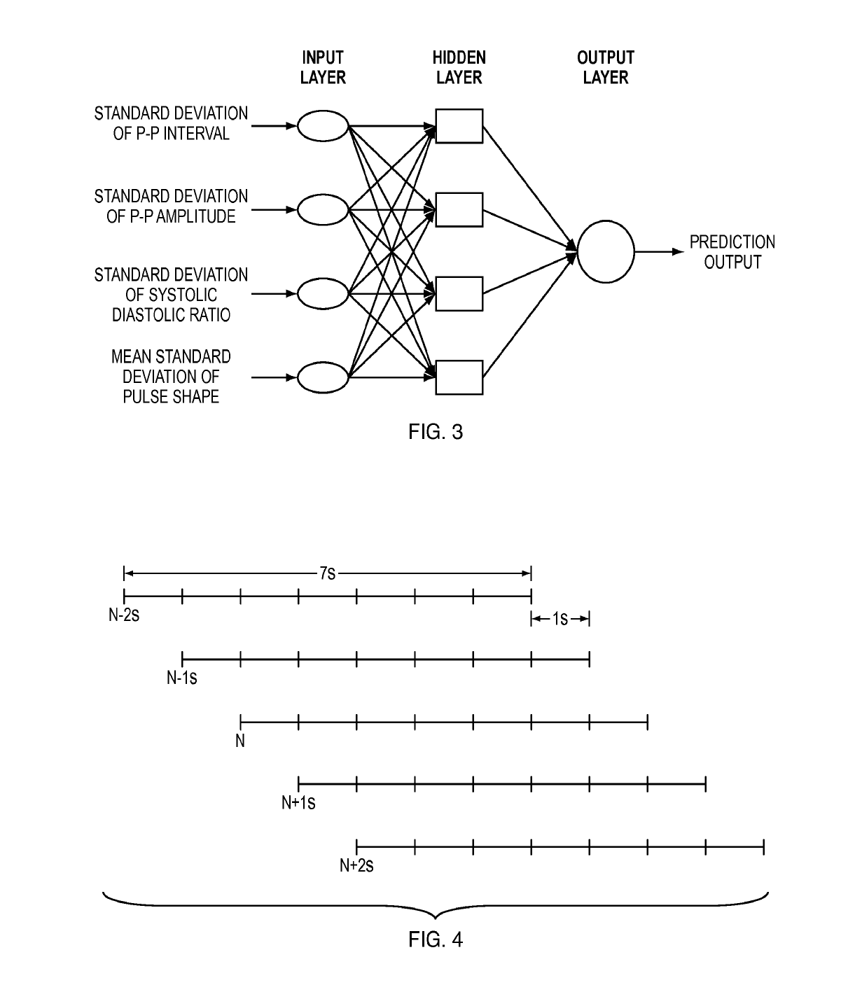

1) Training phase: A flow chart of the training phase in the SVM-based MNA detection algorithm is shown in FIG. 2. The SVM takes the parameter values of clean and corrupted PPG segments as a training data set, finds the support vectors among the training data set which maximize the margin (or the distance) between different classes, and finally builds a decision boundary. If the estimated decision is different from its known label, the decision is regarded as a training error. A soft-margin SVM is considered, which can set the boundary even when the data sets are mixed and cannot be separated. In the soft-margin SVM algorithm, slack variables are introduced to minimize the training error with maximizing the margin. Soft-margin SVM uses the following equation to find the support vectors.

$$Minimize C {\sum }_{sv = 1}^{N} {δ}_{sv} + \frac{1}{2} 〈 {w}_{s} , {w}_{s} 〉 , \\ \begin{matrix}Subject to {T}_{sv} ( 〈 {w}_{s} , {y}_{sv} 〉 + {b}_{s} ) \geq 1 = {δ}_{sv} for sv = 1 , 2 , … , N \\ = 1 , 2 , … , N , and {δ}_{sv} \geq 0\end{matrix} \qquad ( 7 )$$

where C is regulation parameter, N is the number of vectors, δₛᵥ is the slack variable, wₛ is weight vector and <•,•> is the inner product operation. The Tₛᵥ is the sv^{th} target variable, yₛᵥ is the sv^{th} input vector data, and bₛ is the bias. The SVM decision boundary Fsv is derived as

Fₛᵥ= [symbol] w₂*,y [symbol] +bₛ*=0 (8)

where wₛ* and bₛ* are weight factor and bias, respectively, obtained from Eq. (7) and y is the input point.

By transforming the yₛᵥ and y term to yₛᵥ→Φ(yₛᵥ) and y→Φ(y), the non-linear SVM can be transformed to a linear SVM. For nonlinear SVM, Eq. (7) is modified as

Tₛᵥ( [symbol] wₛ,Φ(yₛᵥ) [symbol] +bₛ)≧1 (9)

To facilitate the operation in nonlinear SVM, a kernel function Kₛ(•,•), which is a dot-product in the transformed feature space as follows, is used,

Kₛ(yₛᵥ,y_{sv′})= [symbol] Φ(yₛᵥ),Φ(y_{sv′}) [symbol] (10)

where sv′=1, 2, . . . , N.

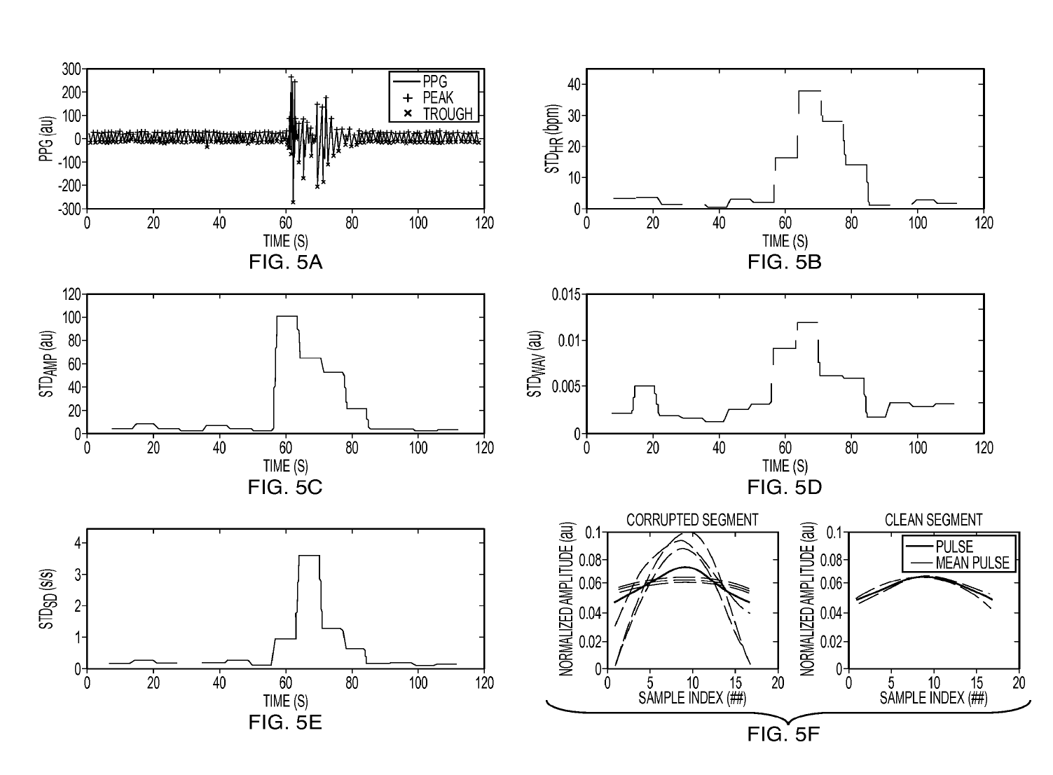

2) Test phase: FIG. 3 shows a flow chart of the test phase in the SVM-based MNA detection algorithm. The PPG data can be partitioned into many 7-second segments. Parameters can be derived from each PPG portion to examine if it is corrupted by motion artifact or not.

Enhancement of MNA Detection by Major Votes

To enhance MNA detection performance, the disclosed algorithm incorporates multiple decisions on a set of neighbor segments in deciding whether a “target” segment is clean or corrupted. Neighbor segment is defined as a segment surrounding a target segment within ±T_{neighbor} seconds. Decision on a neighbor segment is highly likely to be the same as the decision on a target segment since PPG pulses in the neighbor segments are most likely to exhibit similar dynamics to the target segment.

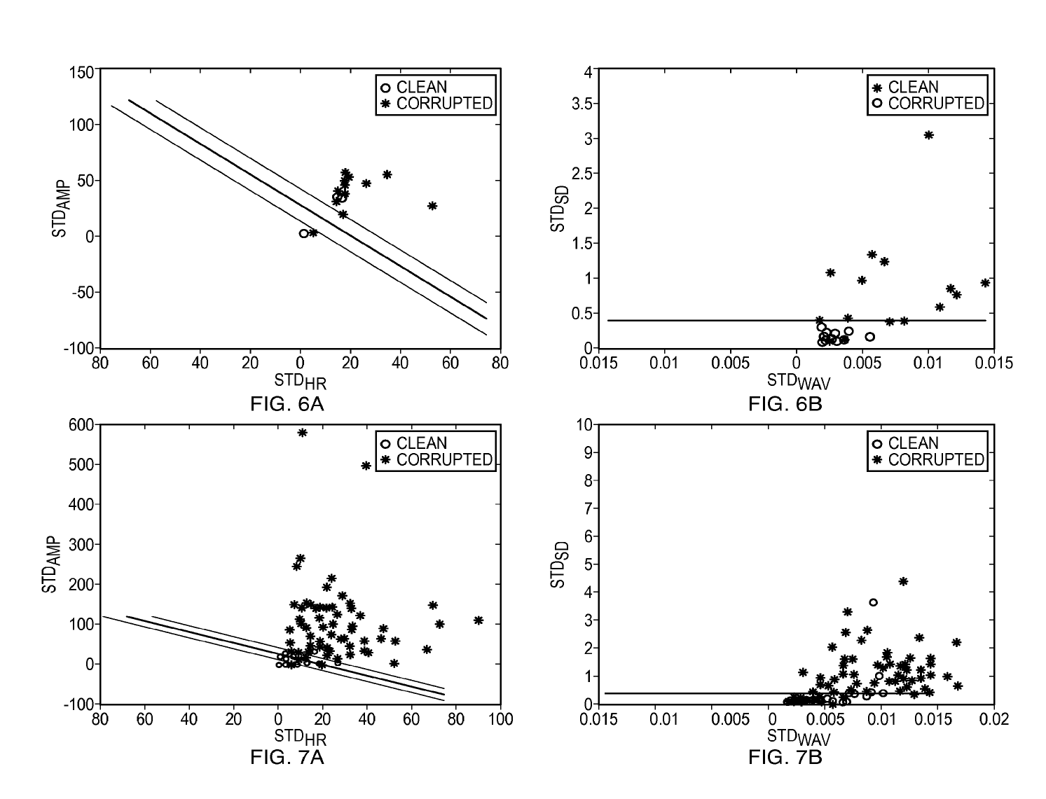

The algorithm gathers the decisions of neighbor segments as well as target segment (see, for example, FIG. 4) and makes a final decision on the target segment based on a majority vote concept (see, for example, Wim H. Hesselink, The Boyer-Moore Majority Vote Algorithm, 7 Nov. 2005, which is incorporated by reference herein in its entirety and for all purposes).

RESULTS—In order to further elucidate these teachings, results of exemplary embodiments are presented hereinbelow.

The performance of the MNA detection algorithm can be evaluated for various types (simulated, laboratory controlled, and daily activities) of motion-corrupted PPGs so as to validate the performance in a wide range of scenarios. For all types of motions, the PPG recordings are divided into 7-second segments since this is determined to be the optimal size among the data length tested from 3-11 seconds (see below PERFORMANCE COMPARISON). Results of the disclosed algorithm are compared with four recently published MNA detection algorithms based on kurtosis (K), Shannon entropy (SE), Hjorth 1 (H1), and Hjorth 2 (H2) metrics, respectively. As performance metrics, classification accuracy, sensitivity, and specificity are considered. In addition, mean HR and SpO2 errors are also investigated as well as detection error ratio.

Reference: Clean Vs. Corrupted

The following are criteria which are adopted to reference PPG segments (clean or corrupted) for each experiment. A visual reference is excluded to avoid subjective decisions by visual inspectors; for subtle MNA, there are large disagreements among visual inspectors. Instead, objective decisions are performed based on controlled corruption start (T_{corr,start}) and end (T_{corr,end}) time points, ECG-derived heart rate (HRECG), PPG-derived heart rate (HRPPG), and SpO2 (SpO2PPG) from PPG signals.

Laboratory controlled data (Forehead and finger):
If more than 85% of a segment is outside of [_{Tcorr,start}, T_{corr,end}], the segment is considered clean. Otherwise, the segment is referenced to be corrupted.
If SpO2(PPG) deviates by 10% from the mean of SpO2(PPG) in a segment, then the segment is referenced to be corrupted.
Successive difference, |diff(HR_{PPG}(i+1)−HR_{PPG}(i))|, from PPG signals is larger than 20 bpm for at least one pulse during a segment, then the segment is referenced to be corrupted.

Daily activity data (Walking and stair-climbing):
Successive difference, |diff(HR_{ECG}(i+1)−HR_{ECG}(i))|, from ECG signals is larger than 20 bpm for at least one pulse during a segment, then the segment is excluded.
If SpO2(PPG) deviates by 10% from the mean of SpO2(PPG) in a segment, then the segment is referenced to be corrupted.
If |diff(HR_{PPG}(i+1)−HR_{PPG}(i))| is larger than 20 bpm for at least one pulse during a segment, then the segment is referenced to be corrupted.
If |HR_{ECG}−HR_{PPG}|<5 bpm during more than 85% of a segment, the segment is considered clean. Otherwise, the segment is referenced to be corrupted.

Table I below describes the number of clean and corrupted PPG segments for each motion type used in the experiment as determined by the criteria defined above.

TABLE I
Numbers of Subjects and Numbers of Clean and
Corrupted Segments per Each Motion Artifact

| | | # of | # of | # of |
|---|---|---|---|---|
| Type | Subtype | Subjects | Clean | Corrupted |
| Simulation | Simulation | N/A | N/A | N/A |
| Laboratory | Finger | 13 | 195 | 105 |
| Controlled | Forehead | 11 | 190 | 110 |
| Daily- | Walking/ | 9 | 125 | 175 |
| Activity | Stair- | | | |
| | climbing | | | |

Classification Accuracy

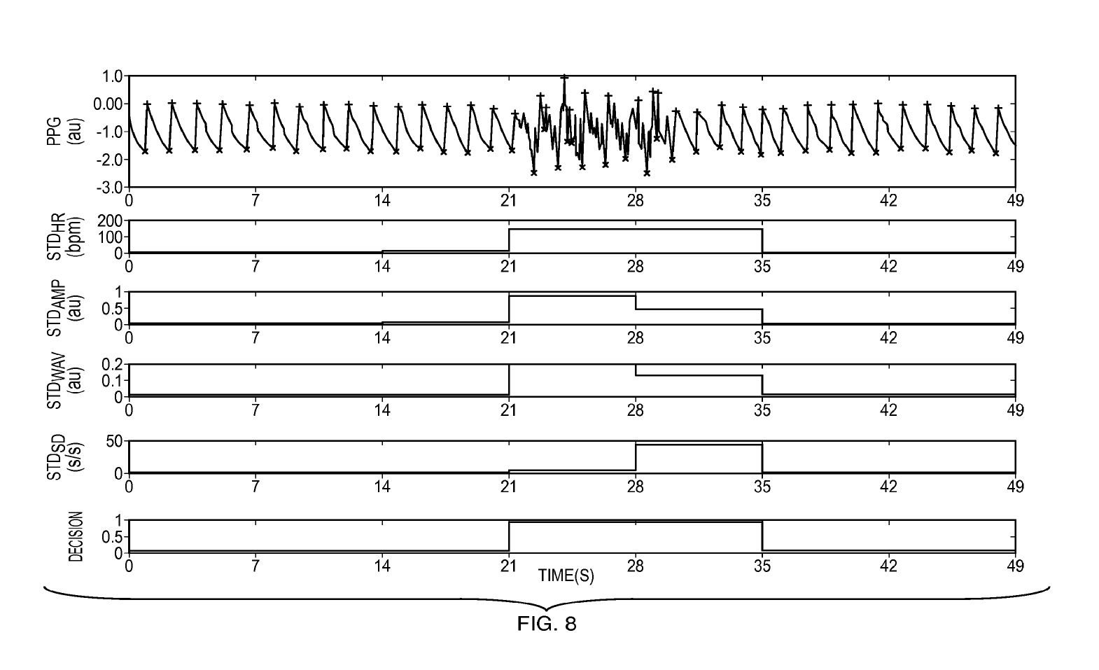

A sample forehead PPG signal and its corresponding parameters calculated segment-by-segment are given in FIG. 5A and FIGS. 5B through 5E, respectively. The normalized sampled corrupt and clean PPGs for mean standard deviation of pulse shape is given in FIG. 5F. The sample signal is corrupted from t=56 to t=85 seconds. Corrupted PPG segments between 56-85 seconds have larger parameter values compared to clean segments between 1-56 seconds and 85-112 seconds.

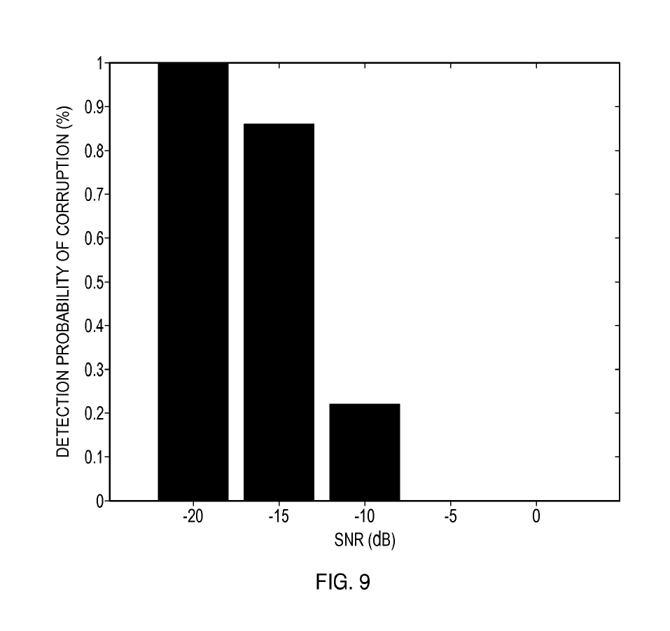

FIGS. 6A and 6B show (STD_{HR},STD_{AMP}) and (STD_{SD},STD_{WAV}) of clean (circle) and corrupted (star) forehead signals, respectively, with corresponding SVM boundaries (black line). To lower computational complexity, a linear kernel is considered for the SVM in the experiment. Regularization parameter value (C) of the linear kernel SVM is optimized in terms of minimizing the training error rate. A 11-fold cross-validation and grid search (C={10⁻³, 10⁻², 10⁻¹, 1, 10¹, 10², 10³})) is adopted, which is widely used to determine C.

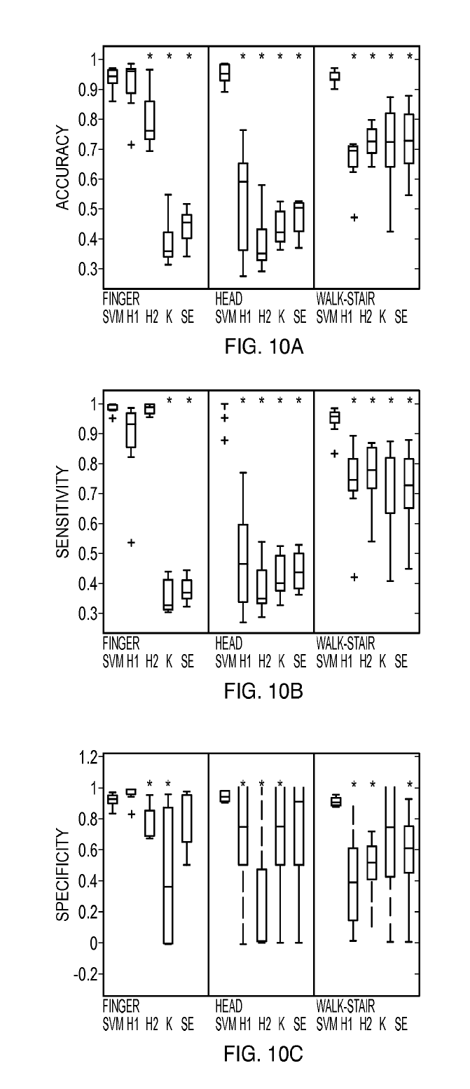

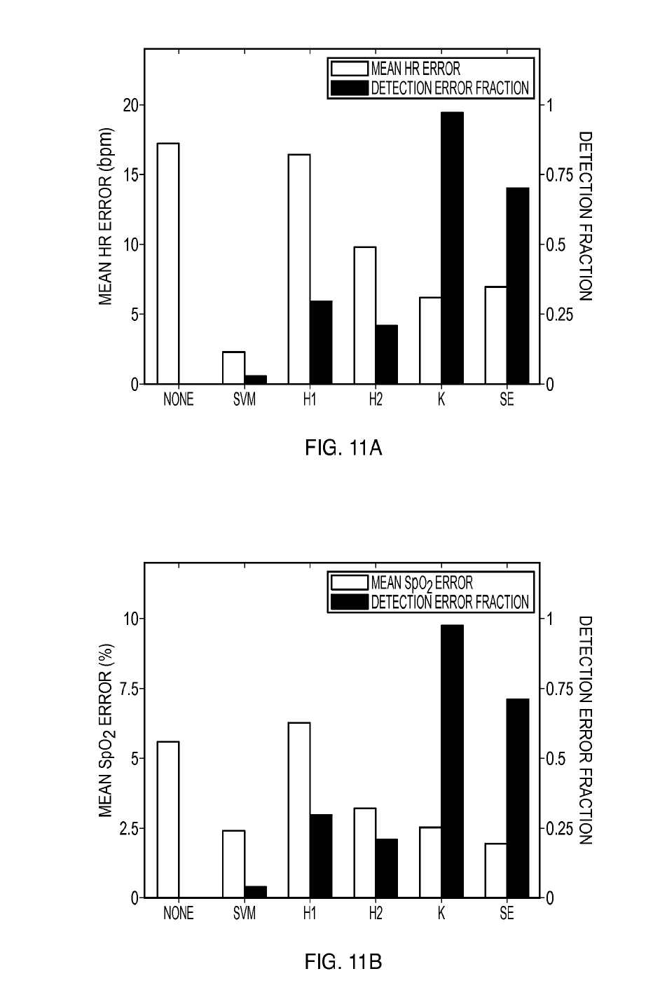

FIG. 7 shows classification results by the SVM boundaries obtained from FIG. 6. FIG. 8 shows a representative PPG signal with detected peaks (red) along with the corresponding statistical parameter values. Note the corrupted PPG signal interval between 21 to 31 seconds. The discrepancy between corrupted and clean portions is reflected by parameters STD_{HR}, STD_{AMP}, STD_{SD}, and STD_{WAV}. The parameter values from the corrupted PPG segments exhibit larger variability and consequently have higher standard deviation value compared to those from clean data segments. The STD_{HR}, STD_{AMP}, and STD_{WAV}, have large values between 21-35 seconds (see FIGS. 8B-8D), while STD_{SD} has large value only between 21-28 seconds (see FIG. 8E). Using SVM with these parameter values, the disclosed algorithm correctly discriminated MNA corrupted segment between 21-35 seconds (see FIG. 8F). Table II below presents C for finger, forehead, and walking/stair-climbing data. The disclosed algorithm is tested to different segment lengths varying from 3 to 11 seconds and calculated their mean classification accuracies, which are provided in below Table III. Among the different data segment lengths tested, the 7-second segment provided the highest classification accuracies for all data: finger, forehead and walking/stair-climbing PPG signals. Accuracy, specificity, and sensitivity for each dataset are presented in Table IV. On average, the SVM performance using the 7-second segment showed a 93.9% accuracy, 92.4% specificity, and 94.3% sensitivity.

TABLE II
C obtained by 9 fold cross-validation and grid search method

| Type | Subtype | C |
|---|---|---|
| Simulation | Simulation | 100 |
| Laboratory | Finger | 1000 |
| Controlled | Forehead | 1 |
| Daily- | Walking/ | 0.01 |
| Activity | Stair- | |
| | climbing | |

TABLE III
Detection accuracy (Mean ± SD) for varying window length

| | Window Length | | | | |
|---|---|---|---|---|---|
| Type | 3 | 5 | 7 | 9 | 11 |
| Finger | 0.883 ± 0.042 | 0.906 ± 0.035 | 0.944 ± 0.033 | 0.944 ± 0.033 | 0.875 ± 0.042 |
| Forehead | 0.883 ± 0.023 | 0.880 ± 0.027 | 0.934 ± 0.035 | 0.856 ± 0.045 | 0.805 ± 0.044 |
| Walk-Stair | 0.813 ± 0.033 | 0.871 ± 0.039 | 0.937 ± 0.026 | 0.867 ± 0.052 | 0.856 ± 0.056 |
| Climbing | | | | | |

TABLE IV
Detection accuracy, specificity and sensitivity
(Mean ± SD) for 7-seconds segment

| Type | Accuracy | Specificity | Sensitivity |
|---|---|---|---|
| Finger | 94.4 ± 3.3 | 94.7 ± 4.5 | 94.7 ± 3.4 |
| Forehead | 93.4 ± 3.5 | 96.7 ± 3.0 | 88.8 ± 7.9 |
| Walking/ | 93.7 ± 2.7 | 91.4 ± 2.0 | 93.9 ± 5.0 |
| Stair- | | | |
| climbing | | | |

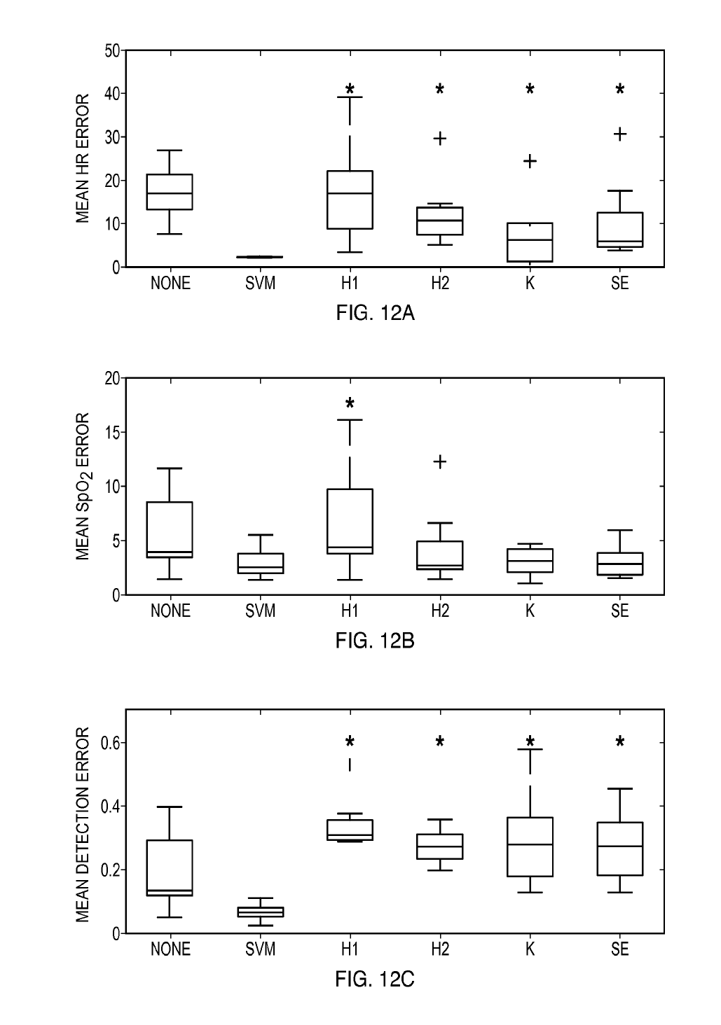

To evaluate the sensitivity of our MNA detection algorithm to noise, Gaussian white noise (GWN) of varying signal-to-noise (SNR) levels is added to a representative non-MNA corrupted PPG signal. For each SNR, 50 independent clean PPG signal. As shown in FIG. 9, the PPG signals with a SNR below −10 dB are detected as corrupted data with our algorithm. For a SNR of −20 dB, every segment is detected as corrupted.

Performance Comparison of MNA Detection Algorithms

The disclosed algorithm is compared with other artifact detection methods based on H1, H2, K and SE since these methods have been shown to provide good detection accuracies. The H1 and H2 parameters represent the central frequency and half of bandwidth, respectively, and are defined as follows:

$${H}_{1} = \sqrt{\frac{{\overline{v}}_{2} ( n )}{{\overline{v}}_{0} ( n )}} and {H}_{2} = \sqrt{\frac{{\overline{v}}_{4} ( n )}{{\overline{v}}_{2} ( n )} - \frac{{\overline{v}}_{2} ( n )}{{\overline{v}}_{0} ( n )}}$$

where v̅ᵢ(n)=∫₋ₙⁿvⁱS_{y}_{_{pDC}}(e^{Jν})dv. Here, S_{y}_{_{pDC}}(e^{Jν}) is the power spectrum of signal y_{pDC}(n).

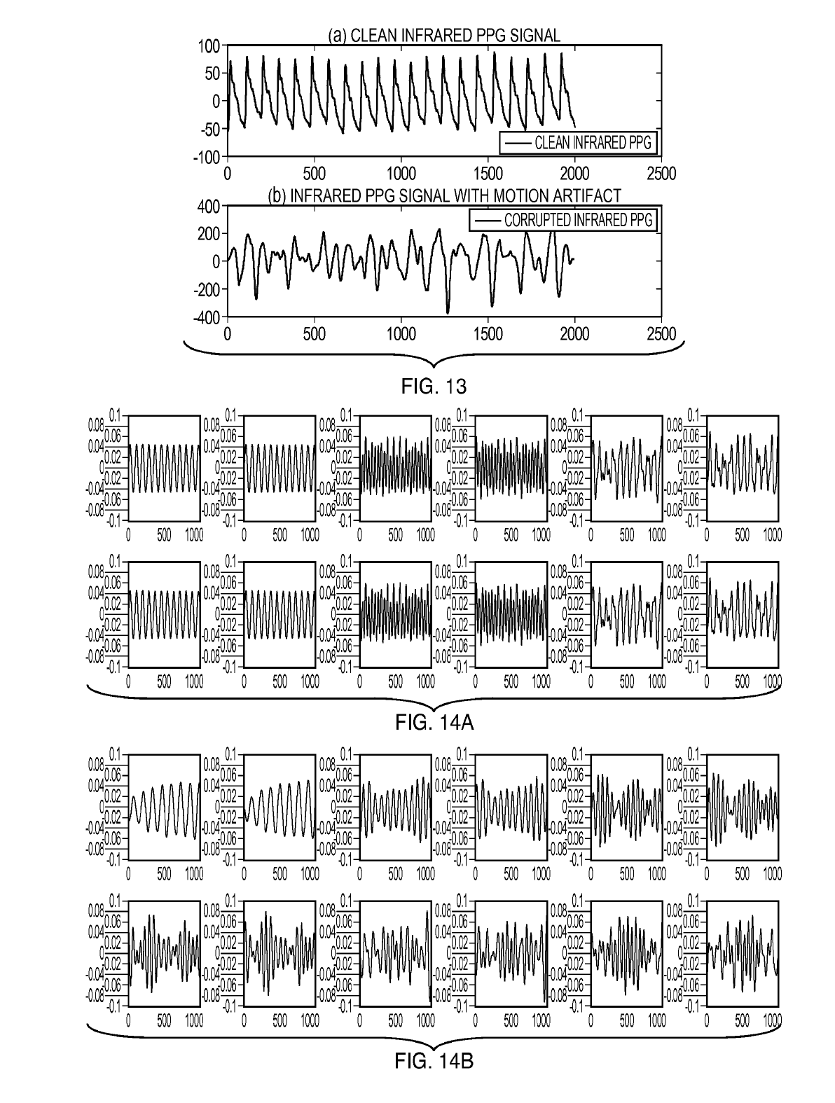

For a fair comparison, all detection methods used 7 second data segments. FIGS. 10A-10C compare the medians and 25th and 75th percentiles of detection accuracy, sensitivity, and specificity for all five detection methods for the finger, head and walking/stair-climbing data sets. In general, the disclosed SVM method consistently yields higher performance with a mean accuracy of 94%, sensitivity of 97%, and a specificity of 92%; whereas other methods show fluctuations depending on which datasets are used. In the finger recorded data, H1 yields a slightly higher accuracy than all other methods due to higher specificity, but the detection sensitivity is lower.

Hr and SpO2 Estimation

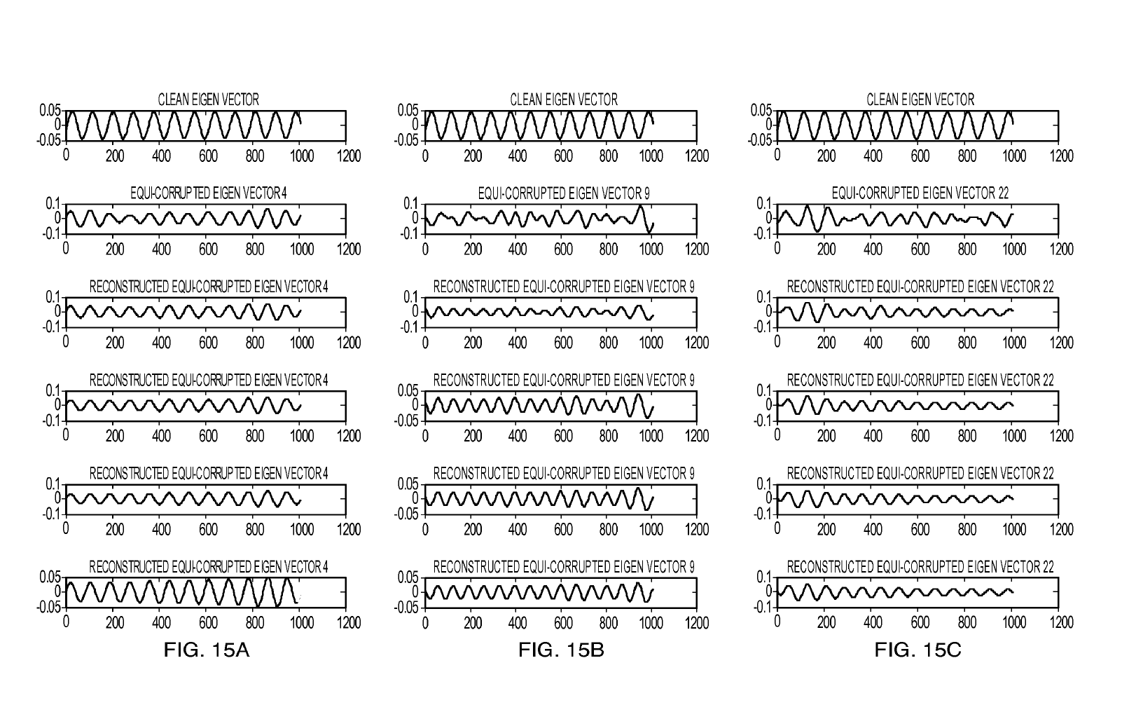

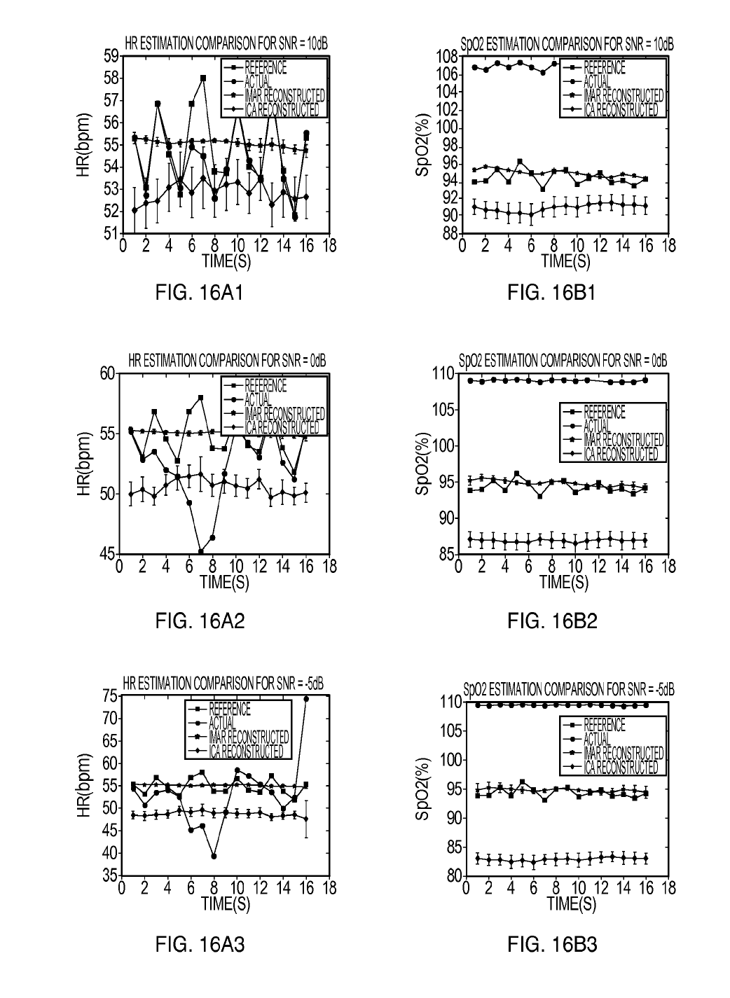

FIG. 11A shows a comparison of the mean HR error and detection error fraction from five MNA detection methods for walking/stair-climbing data. The HR errors are defined by the difference between the estimated HR derived from the PPG and the reference HR readings. Low error values reflect an effective artifact detection algorithm. The disclosed algorithm yields the lowest HR error and detection error fraction as compared with other MNA methods. FIG. 11B shows a comparison of mean SpO2 error and detection error fraction from five MNA detection methods. The SE based detection method shows a lower mean SpO2 error than the disclosed algorithm, but its detection error fraction is very high (>70%), indicating that the error is computed based on only 30% of clean data. On the other hand, the disclosed SVM algorithm resulted in a mean SpO2 error of 2.7 with a detection error of only 6.3%. FIG. 12 shows a comparison of five MNA detection methods in terms of paired-t test results of HR and SpO2 estimation and detection accuracy. On average, the SVM algorithm outperformed the K, SE, H1 and H2 methods with HR errors of 2.3 bpm, SpO2 errors of 2.7% and detection error fraction of 6.3%.

### DISCUSSION

Robust real-time MNA detection algorithms for raw PPG signals have been elusive to date. The disclosed MNA detection algorithm has been designed based on four parameters: (a) standard deviation of peak-to-peak intervals (b) standard deviation of peak-to-peak amplitudes (c) standard deviation of systolic and diastolic time ratios, and (d) mean-standard deviation of pulse shapes. The disclosed MNA algorithm is compared to other well-established MNA detection methods, using the 7-second data segment as this length has been determined to provide the optimal classification accuracy.

The results demonstrate that the disclosed SVM-based MNA detection algorithm has offered higher classification accuracy as well as lower HR and SpO2 errors compared to the conventional detection methods. The paired-t test is performed to determine whether there is a significant difference between classification errors obtained from the disclosed SVM approach compared with other known methods. For the finger recorded PPG segments, FIG. 10A indicates that the mean classification accuracy is significantly different (p<0.05 at 95% CI) between the disclosed SVM method and other methods, except for H1. On the other hand, all other methods are significantly different from the disclosed SVM method for forehead and walking/stair-climbing PPG data. FIGS. 11A and 11B summarizes paired-t test results for HR and SpO2 estimations as well as detection accuracy. As shown in FIGS. 12A-12C, SVM is significantly different from H1, H2, K, and SE in terms of HR estimation and detection accuracy (see FIGS. 12A and 12C), while SpO2 derived from the SVM method is significantly different from only H1 (see FIG. 12B).

The disclosed MNA detection algorithm coded with Matlab (2012a) takes only 7 ms on an Intel Xeon 3.6 GHz computer for the 7-second data segment. Hence, the disclosed algorithm is real-time realizable especially when it is coded in either C or C++. The disclosed computational MNA detection algorithm has provided high HR and SpO2 estimation accuracy as well as classification accuracy. Moreover, the disclosed algorithm shows significantly better performance than some well-cited methods with good detection accuracy. Another key advantage of the disclosed algorithm is that it is able to detail with a near pinpoint accuracy when MNA starts and ends. The other four methods fare poorly when compared to the disclosed algorithm in detecting the start and end time of the MNA. The potential for the method disclosed in this work to have practical applications is high, and the integration of the algorithm described with a pulse oximeter device may have significant implications for real-time clinical applications and especially for ambulatory monitoring of vital signs.

Part II—Motion and Noise Artifacts Removal

In these teachings, a PPG signal can be reconstructed from those portions of data that have been identified to be corrupted using the algorithm detailed hereinabove. The fidelity of the reconstructed signal is determined by comparing the estimated SpO2 and heart rate (HR) to reference values. In addition, the reconstructed SpO2 and HR values obtained via the ICA are compared to those obtained by the method disclosed herein. The ICA results are chosen as the point of comparison, because ICA has recently been shown to provide accurate reconstruction of corrupted PPG signals.

Experimental Protocol and Preprocessing

In order to further elucidate the teachings presented hereinbelow, data for exemplary embodiments was collected. Three sets of data are collected from healthy subjects recruited from the student community of Worcester Polytechnic Institute (WPI). This study is approved by WPI's institutional review board and all the subjects give informed consent before data recording.

In the first experiment, eleven healthy volunteers are asked to wear a forehead reflectance pulse oximeter developed in the lab along with a reference Masimo Radical (Masimo SET®) finger transmittance pulse oximeter. PPG signals from the forehead sensor and reference (HR) derived from a finger pulse oximeter are acquired simultaneously. The HR and SpO2 signals are acquired at 80 Hz and 1 Hz, respectively. After baseline recording for 5 minutes without any movement (i.e. clean data), motion artifacts are induced in the PPG data by the spontaneous movements in both horizontal and vertical directions of the subject's head while the right middle finger is kept stationary. Subjects are directed to introduce the motions for specific time intervals that determined the percentage of noise within each 1 minute segment, varying from 10 to 50%. For example, if a subject is instructed to make left-right movements for 6 seconds, a 1 minute segment of data would contain 10% noise.

The second dataset includes finger-PPG signals from the same 9 healthy volunteers in an upright sitting posture using an infrared reflection type PPG transducer (TSD200). An MP1000 pulse oximeter (commercially available from BIOPAC Systems Inc., CA, USA) is also used to acquire finger PPG signals at 100 Hz. One pulse oximeter of each model is placed on the same hand's index finger (one model) and middle finger (the other model) simultaneously. After baseline recording for 5 minutes without any movement (i.e. clean data), motion artifacts are induced in the PPG data by the left-right movements of the index finger while the middle finger is kept stationary to provide a reference. Similar to the first dataset, motion is induced at specific time intervals corresponding to 10 to 50% corruption duration in 1 minute segments, i.e. the controlled movement is carried out five times per subject.

The third dataset includes data measurements from 9 subjects with the PPG signal recorded from the subjects' forehead using a custom sensor simultaneously with the reference ECG, HR and SpO2 from a Holter Monitor at 180 Hz and Masimo (Rad-57) pulse oximeter at 0.5 Hz respectively. The reference pulse oximeter provided HR and SpO2 measured from the subject's right index finger, which is held steadily to their chest. The signals are recorded while the subjects are going through sets of walking and climbing up and down flights of stairs for approximately 45 min.

Once data are acquired, PPG signals from all three experiments outlined above are preprocessed offline using, for example, Matlab (MathWorks, R2012a). The PPG signals are filtered using a zero-phase forward-reverse 4th order IIR band-pass filter with cutoff frequency 0.5-12 Hz.

Motion Artifact Removal

To reconstruct the artifact-corrupted portion of the PPG signal that has been detected using the support vector machine approach provided herein, a hybrid procedure is developed, using Iterative Singular Spectrum Analysis (ISSA) and a frequency matching algorithm. Henceforth, the combined procedures is referenced as the iterative motion artifact removal (IMAR) algorithm.

A method of these teachings includes a method for removal of motion and noise artifacts (MNA) present in a segment of PPG data, by the steps of: (a) for each one segment from a segment of PPG data in which presence of motion and noise artifacts has been previously detected, referred to as a corrupted segment, and a most prior adjacent segment of PPG data in which motion and noise artifacts are not detected, referred to as a clean segment, performing the following: (a1) assemble a data transition matrix, each row of the data transition matrix being a vector of a predetermined length, a number of vectors being equal to a number of samples in a segment for which the data transition matrix is assembled minus the predetermined length and plus one; a starting value of each vector being displaced by one sample from a previous vector, resulting in the data transition matrix having a number of columns equal to the predetermined length and a number of rows equal to the number of vectors; (a2) obtain eigenvectors and eigenvalues for the data transition matrix, resulting in eigenvectors and eigenvalues for the corrupted segment and eigenvectors and eigenvalues for the clean segment; (b) sorting the eigenvalues for the corrupted segment from largest to smallest; and sorting the eigenvalues for the clean segment from largest to smallest; (c) retaining only a top predetermined percentage of the eigenvalues for the corrupted segment and the eigenvalues for the clean segment; (d) replacing the eigenvalues for the corrupted segment with the eigenvalues for the clean segment, where only the top predetermined percentage of the eigenvalues and corresponding eigenvectors have been retained; (e) retaining only eigenvectors for the corrupted segment and eigenvectors for the clean segment that have data in a predetermined frequency range; (f) discarding eigenvectors for the corrupted segment that have different frequencies from the eigenvectors for the clean segment; (g) obtaining the data transition matrix for the corrupted segment from the eigenvalues and eigenvectors of the corrupted segment and the data transition matrix for the clean segment from the eigenvalues and eigenvectors of the clean segment; (h) repeating steps (a2) to (g) until a predetermined convergence criterion is satisfied; and (i) reconstructing, after the predetermined convergence criterion is satisfied, the corrupted segment from the data transition matrix for the corrupted segment using replaced eigenvalues and retained eigenvectors. The predetermined length is less than one half of a number of samples in the segment for which the data transition matrix is assembled and is larger than a ratio of a sampling frequency to a lowest frequency in said segment being considered. The predetermined convergence criterion is a difference between a discarding metric for the corrupted segment reconstructed from the data transition matrix using replaced eigenvalues and retained eigenvectors and a discarding metric for the clean segment, the discarding metric being a sum of absolute values of signal components divided by a length metric for the signal components. The predetermined frequency range is a heart rate range of PPG data. The predetermined frequency range includes frequencies greater than 0.66 Hz and less than 3 Hz. The top predetermined percentage is a top 5%. In this method, the presence of motion and noise artifacts had been previously detected using the method previously described.

Singular Spectrum Analysis (SSA)

The SSA is composed of two stages: A) singular decomposition and B) spectral reconstruction. The former is the spectral decomposition or eigen-decomposition of the data matrix whereas the latter is the reconstruction of the signal based on using only the significant eigenvectors and associated eigenvalues. The assumption is that given a relatively high signal-to-noise ratio of data, significant eigenvectors and associated eigenvalues represent the signal dynamics and less significant values represent the MNA components.

The calculation of the singular stage of the SSA includes two steps: i) embedding followed by ii) singular value decomposition (SVD). In essence, these procedures decompose the data into signal dynamics including trends, oscillatory components, and MNA. The spectral stage of the SSA algorithm also includes two steps: i) grouping and ii) diagonal averaging. These two procedures are used to reconstruct the signal dynamics but without the MNA components. In the following section, we detail all four steps in the SSA algorithm.

Singular Decomposition—Embedding

Assume there is a nonzero real-value time series of length N samples, i.e., x=(x₁, x₂, . . . , x_{N}). In the embedding step, window length fₛ/f₁<L<N/2 is chosen to embed the initial time series, where fₛ is the sampling frequency and f₁ is the lowest frequency in the signal. The time series X is mapped into the L lagged vectors, x=(xᵢ, xᵢ₊₁, . . . , x_{i+L-1}) for i=1, . . . , K, where K=N−L+1. The result is the trajectory data matrix Tₓ or vector Xᵢ that is each row of Tₓ for i=1, . . . , K.

$${T}_{x} = [ \begin{matrix}{X}_{1} \\ {X}_{2} \\ ⋮ \\ {X}_{K}\end{matrix} ] = [ \begin{matrix}{x}_{1} & {x}_{2} & … & {x}_{L} \\ {x}_{2} & {x}_{3} & … & {x}_{L + 1} \\ ⋮ & ⋮ & ⋱ & ⋮ \\ {x}_{K} & {x}_{K + 1} & … & {x}_{N}\end{matrix} ] \qquad ( 11 )$$

From Eq. 11, it is evident that the trajectory matrix, Tₓ, is a Hankel matrix.

Singular Decomposition—Singular Value Decomposition

The next step is to apply the SVD to the trajectory matrix Tₓ which results in eigenvalues and eigenvectors of the matrix T_{X}Tₓ^{T} where Tᵢ for i=1, . . . , L can be defined as T=USV^{T}. Uᵢ for 1<i<L is a K×L orthonormal matrix. Sᵢ for 1<i<L is a diagonal matrix and vᵢ for 1<i<L is an square orthonormal matrix, which is considered the principle component. In this step, Tₓ has L many singular values which are \sqrt{λᵢ}>\sqrt{λ₂}>, . . . , \sqrt{λ_{L}}. Thus, the i^{th} eigentriple of Tᵢ can be written as Uᵢ×\sqrt{λᵢ}×Vᵢ^{T} for i=1, 2, . . . , d, in which d=max(i: \sqrt{λᵢ}>0) is the number of nonzero singular values of Tₓ. Normally, every harmonic component with a different frequency produces two eigentriples with similar singular values. So the trajectory matrix Tₓ can be denoted as

$$\begin{matrix}{T}_{x} = {T}_{1} + {T}_{2} + … + {T}_{d} \\ = {U}_{1} \sqrt{{λ}_{1}} {V}_{1}^{T} + … + {U}_{d} \sqrt{{λ}_{d}} {V}_{d}^{T} \\ = {\sum }_{i = 1}^{d} {U}_{i} \sqrt{{λ}_{i}} {V}_{i}^{T}\end{matrix} \qquad ( 12 )$$

Projecting the time series onto the direction of each eigenvector yields the corresponding temporal principal component (PC).

Spectral Reconstruction

The reconstruction stage has two steps: i) grouping and ii) diagonal averaging. First, the subgroups of the decomposed trajectory matrices are grouped and then a diagonal averaging step is needed so that a new time series can be formed.

Spectral Reconstruction—Grouping

The grouping step of the reconstruction stage decomposes the L×K matrix Tᵢ into subgroups according to the trend, oscillatory components, and MNA dynamics. The grouping step divides the set of indices {1, 2, . . . , d} into a collection of m disjoint subsets of I={Iₗ, . . . , Iₘ}. Thus, T_{I} corresponds to the group I={Iₗ, . . . , Iₘ}. Tₗ_{ᵢ} is a sum of Tⱼ, where jεIⱼ. So Tₓ can be expanded as

$${T}_{x} = \overbrace{{T}_{1} + … + {T}_{L}}^{SVD} = \overbrace{{T}_{{I}_{1}} + … + {T}_{{I}_{m}}}^{Grouping} \qquad ( 13 )$$

Spectral Reconstruction—Diagonal Averaging

In the final step of analysis, each resultant matrix, Tₗᵢ, in Eq. (13) is transformed into a time series of length N. We obtain the new Hankel matrices \tilde{X}⁽ⁱ⁾ by averaging the diagonal elements of the matrix Tₗ_{ᵢ}. Let H be denoted as the Hankel operator. So that we obtain the Hankel matrix \tilde{X}⁽ⁱ⁾=HTₗ_{ᵢ} for i=1, . . . , m. Under the assumption of weak separability and applying the Hankel procedure to all matrix components of Eq. (13), we obtain the following expansion

X=\tilde{X}⁽¹⁾+\tilde{X}⁽²⁾+ . . . +\tilde{X}^{(M)} (14)

We can assert that \tilde{X}⁽¹⁾ is related to the trend of the signal; however, harmonic and noisy components do not necessarily follow the order of \sqrt{λ₁}>\sqrt{λ₂}> . . . >\sqrt{λ_{M}}.

Iterative Motion Artifact Removal Based on SSA

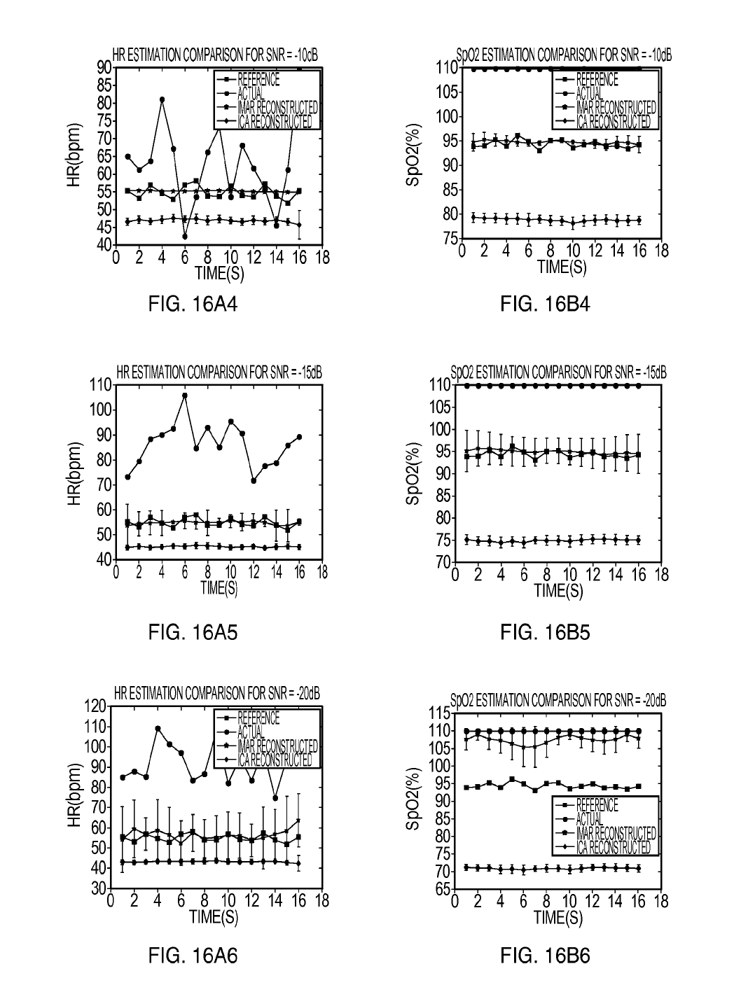

In order to reconstruct the MNA corrupted segment of the signal, an iterative motion artifact removal approach based on SSA is explained in the last section. The ultimate goodness of the reconstructed signal is determined by the accuracy of the estimated SpO2 and HR values. The top and bottom panels of FIG. 13 show clean and MNA corrupted signals, respectively.

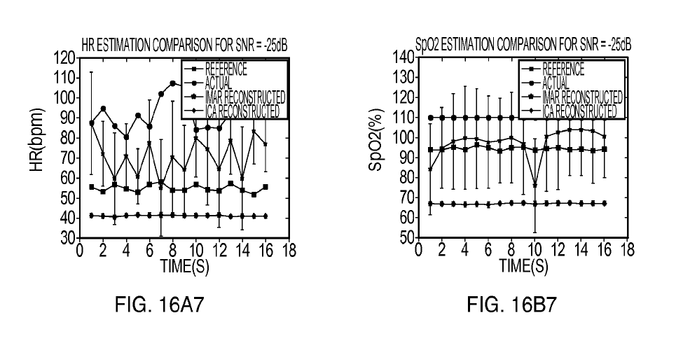

FIGS. 14A and 14B show the first 12 eigenvectors of the clean and MNA corrupted data as shown in FIG. 13, respectively. The most important part of the SSA is to choose the proper eigenvector components for reconstruction of the signal. Under the assumption of high SNR, the normal practice is to select only the largest eigenvalues and associated eigenvectors for signal reconstruction. However, most often it is difficult to determine the demarcation of the significant from non-significant eigenvalues. Further, the MNA dynamics can overlap with the signal dynamics, hence, choosing the largest eigenvalues does not necessarily result in an MNA-free signal.

To overcome the above limitations, the SSA approach is modified. The first step of the modified SSA involves computing singular value decomposition on both a corrupted data segment and its most prior adjacent clean data segment. Under the assumption of a high SNR of the data, the second step is to retain only the top 5% of the eigenvalues and their associated eigenvectors. The third step is to replace the corrupted segment's top 5% eigenvalues with the clean segment's eigenvalues. The fourth step is to further limit the number of eigenvectors by choosing only those eigenvectors that have heart rates between for both the clean and noise corrupted data segments. The two extreme heart rates are chosen so that they account for possible scenarios that one may encounter with low and high heart rates. With the remaining candidate eigenvectors resulting from step four, non-significant eigenvectors are further pruned by performing frequency matching of the noise corrupted eigenvectors to those of the clean data segment's eigenvectors, in the fifth step. Only those eigenvectors' frequencies that match to those of the clean eigenvectors are retained from the pool of eigenvectors remaining from step four. For the remaining eigenvector candidates, iterative SSA is performed to further reduce MNA and match the dynamics of the clean data segments' eigenvectors for the final step. For each iteration, the standard SSA algorithm is performed. Experience shows that convergence is achieved within 4 iterations.

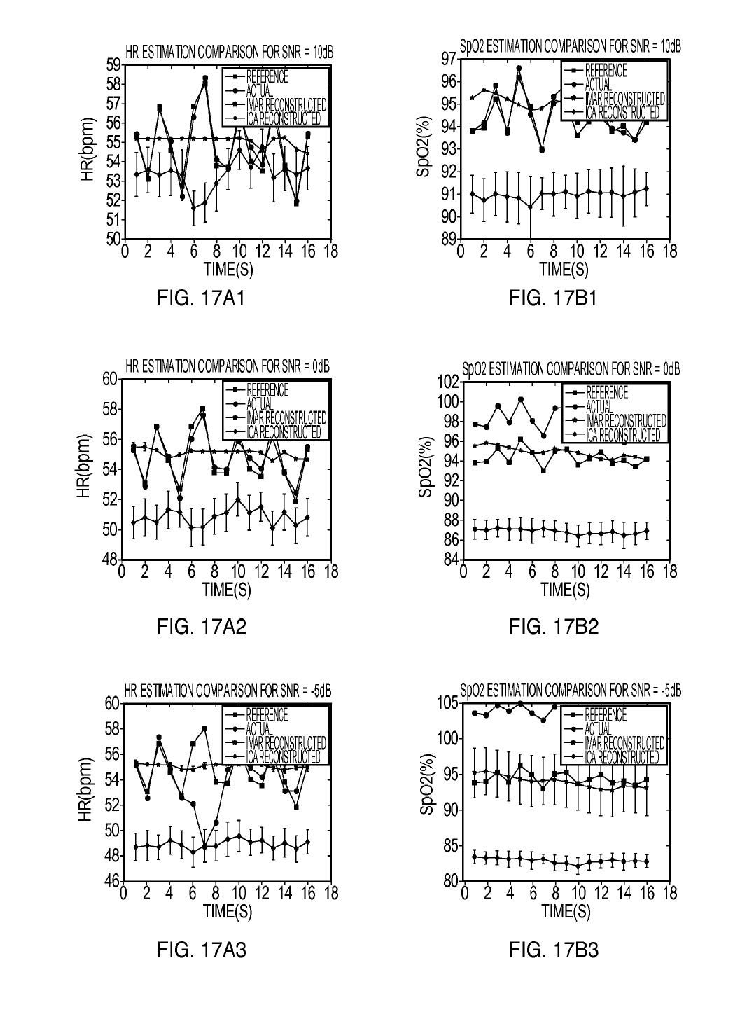

FIGS. 15A-15C show examples of the iterative SSA procedure applied to candidate eigenvectors that have resulted from step four of the procedure for the modified SSA algorithm. Note that there may be several eigenvectors remaining after the fifth step, hence, these examples show an iterative SSA procedure performed on a particular set of candidate eigenvectors that may match most closely to an eigenvector of a clean data segment. The row of panels in FIG. 15A represents one of the eigenvectors of the clean signal. The row of panels in FIG. 15B represents the MNA corrupted signal's candidate eigenvectors which have the same frequency as that of the clean signal's eigenvector. The row of panels in FIG. 15C represents the candidate eigenvectors after they have gone through four successive iterations of the SSA algorithm. For this portion of the SSA algorithm, SVD is performed on the trajectory matrix of Eq. (11) created from the candidate eigenvector and then reconstruct the eigenvectors based on SSA using only the first 3 largest eigenvalues obtained from the SVD. This process repeats iteratively until the shape of the reconstructed eigenvector closely resembles one of the clean eigenvectors with the same frequency. It can be seen from FIGS. 15A-15C that after 4 iterations the result shown in the panel of FIG. 15A corresponds most closely to the clean signal's eigenvector, hence, this eigenvector is selected rather than the eigenvectors shown in panels in FIGS. 15B and 15C. The discarding metric (DM) is calculated at each iteration and the value is compared to the DM value of the corresponding clean component. The DM is calculated according to:

DM=Σ|u|/L(u) (15)

where u is the signal component, and |•|, L(•) are absolute operator and component length, respectively. The entire procedure for the modified SSA algorithm is summarized in TABLE V.

TABLE V
Iterative Motion Artifact Removal (IMAR) Procedure

Assumption -Heart rate and SpO₂ do not change abruptly and are
stationary within the short data segment.
Application - Offline Motion Artifact Removal
Objective - Reconstruction of corrupted PPG segment for the purpose of
estimating heart rates and SpO₂.
Routine
Step 1. First, compute SVD on both corrupted data segments and their
most prior adjacent clean data segments
Step 2. Next, keep the top 5% of the clean and corrupted components,
based on the eigenvalues being sorted from largest to smallest.
Step 3. Replace the corrupted eigenvalues with corresponding clean
eigenvalues.
Step 4. Among the clean and corrupted components, only choose those
with frequency within the heart rate frequency range of 0.66 < Fₛ < 3 Hz.
Step 5. Apply frequency matching to discard those corrupted components
(from Step 4) with different frequencies compared to clean components'
frequencies.
Step 6. Remove corruption from each component obtained from Step 5 by
applying the basic SSA algorithm iteratively.
6. a. Calculate the discarding metric for components achieved from SSA
iterations and their counterpart clean components from Eq. 15.
6. b. Select those processed components with the closest DM and
frequency value to the corresponding clean component's DM and
frequency value.
Step 7. Finally, reconstruct the corrupted PPG segment based on the
components achieved from Step 6.

Results—Noise Sensitivity Analysis

To validate the disclosed IMAR procedure, different SNR levels of Gaussian white noise (GWN) and colored noise are added to an experimentally collected clean segment of PPG signal. One purpose of the simulation is to quantitatively determine the level of noise that can be tolerated by the algorithm. Seven different SNR levels ranging from 10 dB to −25 dB are considered. For each SNR level, 50 independent realizations of GWN and colored noise are added separately to a clean PPG signal. The Euler-Maruyama method is used to generate colored noise.

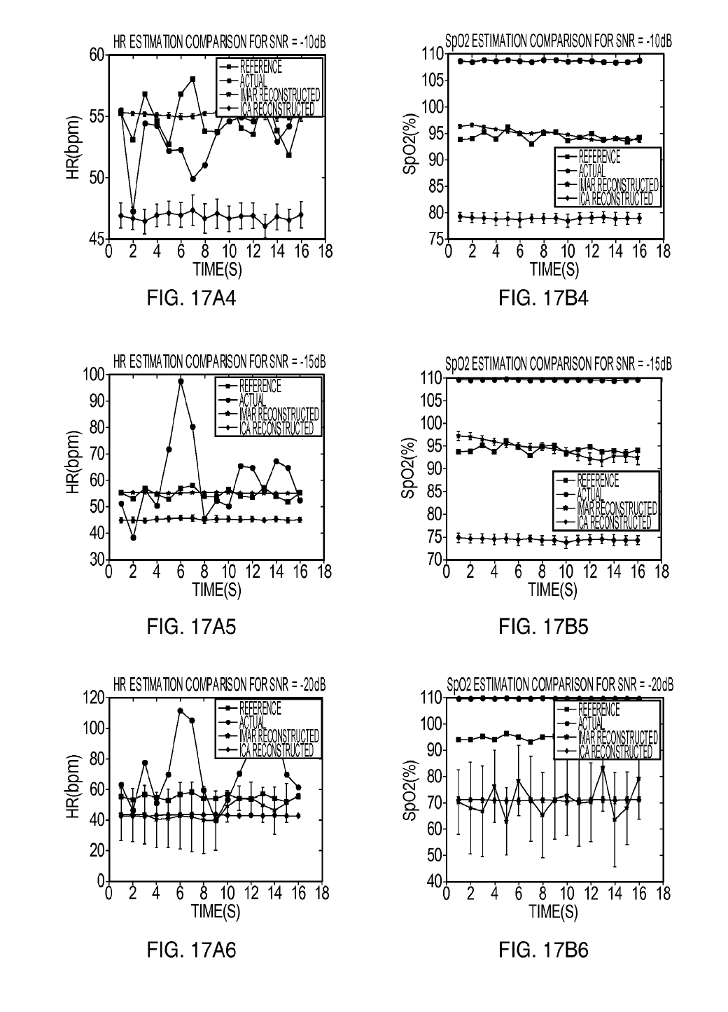

FIG. 16 shows the results of these simulations with additive GWN. The left panels (FIGS. 16A1 to 16A7) show pre- and post-reconstruction HR in comparison to the reference HR; the right panels (FIGS. 16B1 to 16B7) show the corresponding comparison for SpO2. Below Tables VI and VII show the mean and standard deviation values of the pre-(2nd column) and post-reconstruction (4th column), and the reference (3rd column) HR and SpO2 values, respectively for all SNR. The last columns of Tables II and III also show the estimated HR and SpO2 values obtained by the ICA method. As shown in FIG. 16 and Tables VI and VII, the reconstructed HR and SpO2 values using our IMAR approach are found to be not statistically different when compared to the reference values for all SNR except for −20 and −25 dB. However, the ICA method fails and significantly different values are obtained to those of the reference HR and SpO2 values when the SNR is lower than −10 dB.

TABLE VI
Comparison & Statistical Analysis of HR Estimations from IMAR-
reconstructed PPG for Different Levels of Additive White Noise.

| | Head | Finger HR | IMAR Recon- | ICA Recon- |
|---|---|---|---|---|
| SNR | HR | (Reference) | structed HR | structed HR |
| (dB) | (mean _{±} std) | (mean _{±} std) | (mean _{±} std) | (mean _{±} std) |
| 10 | 54.80 _{±} 2.08 | 54.81 _{±} 1.81 | 55.05 _{±} 0.15 | 52.86 _{±} 0.44* |
| 0 | 54.80 _{±} 2.72 | 54.81 _{±} 1.81 | 55.05 _{±} 0.14 | 50.58 ± 0.62* |
| −5 | 56.37 _{±} 8.18 | 54.81 _{±} 1.81 | 55.05 _{±} 0.15 | 48.64 _{±} 0.51* |
| −10 | 46.02 _{±} 22.93 | 54.81 _{±} 1.81 | 55.09 _{±} 0.15 | 46.85 ± 0.45* |
| −15 | 121.62 _{±} 69.33 | 54.81 _{±} 1.81 | 54.73 _{±} 0.62 | 45.17 ± 0.28* |
| −20 | 80.08 _{±} 37.69 | 54.81 _{±} 1.81 | 56.49 _{±} 2.69 | 43.08 ± 0.32* |
| −25 | 103.62 _{±} 52.49 | 54.81 _{±} 1.81 | 76.45 _{±} 7.52* | 41.11 ± 0.30* |

*represents p < 0.05.

TABLE VII
Comparison & Statistical Analysis of Estimations
from IMAR-reconstructed PPG for Different
Levels of Additive White Noise.

| | | Finger | IMAR Recon- | ICA Recon- |
|---|---|---|---|---|
| | Head | SpO₂ | structed | structed |
| SNR | SpO₂ | (Reference) | SpO₂ | SpO₂ |
| (dB) | (mean _{±} std) | (mean _{±} std) | (mean _{±} std) | (mean _{±} std) |
| 10 | 106.88 _{±} 0.51 | 94.23 _{±} 0.80 | 94.83 _{±} 0.38 | 90.92 _{±} 0.38* |
| 0 | 108.98 _{±} 0.14 | 94.23 _{±} 0.80 | 94.81 _{±} 0.42 | 86.88 _{±} 0.16* |
| −5 | 109.42 _{±} 0.06 | 94.23 _{±} 0.80 | 94.77 _{±} 0.26 | 82.86 _{±} 0.27* |
| −10 | 109.69 _{±} 0.04 | 94.23 _{±} 0.80 | 94.68 _{±} 0.30 | 78.81 _{±} 0.29* |
| −15 | 109.82 _{±} 0.02 | 94.23 _{±} 0.80 | 94.90 _{±} 0.41 | 74.88 _{±} 0.23* |
| −20 | 109.89 _{±} 0.01 | 94.23 _{±} 0.80 | 107.38 _{±} 1.06* | 70.87 _{±} 0.22* |
| −25 | 109.94 _{±} 0.00 | 94.23 _{±} 0.80 | 97.38 _{±} 7.39* | 66.91 _{±} 0.26* |

*represents p < 0.05.

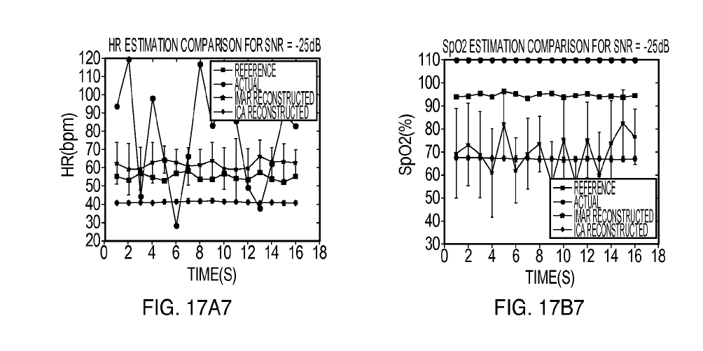

FIG. 17 and below Tables VIII and IX show corresponding results to that of FIG. 16 and Tables VI and VII, but with additive colored noise. Similar to the GWN case, the reconstructed HR and SpO2 values using the disclosed IMAR approach are found to be not significantly different than the reference values for all SNR except for −20 and −25 dB. Moreover, the ICA compares poorly compared to our IMAR as the HR and SpO2 values from the former method are found to be significantly different to the reference values for all SNR.

TABLE VIII
Comparison &nStatistical Analysis of HR Estimations from IMAR-
reconstructed PPG for Different Levels of Additive Colored Noise.

| | | Finger HR | IMAR Recon- | ICA Recon- |
|---|---|---|---|---|
| SNR | Head HR | (Reference) | structed HR | structed HR |
| (dB) | (mean _{±} std) | (mean _{±} std) | (mean _{±} std) | (mean _{±} std) |
| 10 | 54.75 _{±} 1.73 | 54.81 _{±} 1.81 | 55.05 _{±} 0.26 | 53.36 _{±} 0.79 |
| 0 | 55.64 _{±} 2.72 | 54.81 _{±} 1.81 | 55.06 _{±} 0.27 | 50.83 _{±} 0.54* |
| −5 | 55.67 _{±} 2.88 | 54.81 _{±} 1.81 | 55.06 _{±} 0.15 | 48.90 _{±} 0.32* |
| −10 | 51.05 _{±} 8.24 | 54.81 _{±} 1.81 | 55.07 _{±} 0.13 | 46.79 _{±} 0.30* |
| −15 | 61.65 _{±} 32.08 | 54.81 _{±} 1.81 | 55.17 _{±} 0.08 | 45.15 _{±} 0.30* |
| −20 | 73.41 _{±} 47.13 | 54.81 _{±} 1.81 | 45.96 _{±} 5.59* | 42.96 _{±} 0.41* |
| −25 | 66.37 _{±} 40.80 | 54.81 _{±} 1.81 | 61.86 _{±} 2.12* | 41.04 _{±} 0.37* |

*represents p < 0.05.

TABLE IX
Comparison & Statistical Analysis of SpO2 Estimations from IMAR-
reconstructed PPG for Different Levels of Additive Colored Noise.

| | | Finger | IMAR Recon- | ICA Recon- |
|---|---|---|---|---|
| | Head | SpO2 | structed | structed |
| SNR | SpO2 | (Reference) | SpO2 | SpO2 |
| (dB) | (mean _{±} std) | (mean _{±} std) | (mean _{±} std) | (mean _{±} std) |
| 10 | 94.14 _{±} 0.99 | 94.23 _{±} 0.80 | 94.85 _{±} 0.41 | 90.95 _{±} 0.18* |
| 0 | 94.71 _{±} 1.20 | 94.23 _{±} 0.80 | 94.85 _{±} 0.53 | 86.84 _{±} 0.24* |
| −5 | 96.19 _{±} 1.41 | 94.23 _{±} 0.80 | 93.92 _{±} 0.83 | 82.86 _{±} 0.34* |
| −10 | 99.27 _{±} 1.46 | 94.23 _{±} 0.80 | 94.88 _{±} 0.96 | 78.89 _{±} 0.18* |
| −15 | 103.00 _{±} 0.88 | 94.23 _{±} 0.80 | 94.42 _{±} 1.71 | 74.87 _{±} 0.25* |
| −20 | 107.63 _{±} 0.26 | 94.23 _{±} 0.80 | 74.74 _{±} 7.92* | 70.89 _{±} 0.17* |
| −25 | 105.91 _{±} 0.49 | 94.23 _{±} 0.80 | 70.75 _{±} 15.08* | 66.89 _{±} 0.26* |

*represents p < 0.05.

Results—Heart Rate and Estimation from Forehead Sensor

As described above, PPG data are collected under three different experimental settings so that the disclosed approach could be more thoroughly tested and validated. For all three experimental settings, the efficacy of the disclosed IMAR approach for the reconstruction of the MNA-affected portion of the signal is compared with the reference HR and SpO2 values for all experimental datasets.

For the error-free SpO2 estimation, Red and IR PPG signals with clearly separable DC and AC components are required. The pulsatile components of the Red and IR PPG signals are denoted as AC_{Red} and DC_{Red}, respectively, and the “ratio-of-ratio” is estimated as

$$R = \frac{{AC}_{red} / {DC}_{red}}{{AC}_{IR} / {DC}_{IR}} \qquad ( 16 )$$

Accordingly, SpO2 is computed by substituting the R value in an empirical linear approximate relation given by

SpO₂(%)=(110−25R)(%) (17)

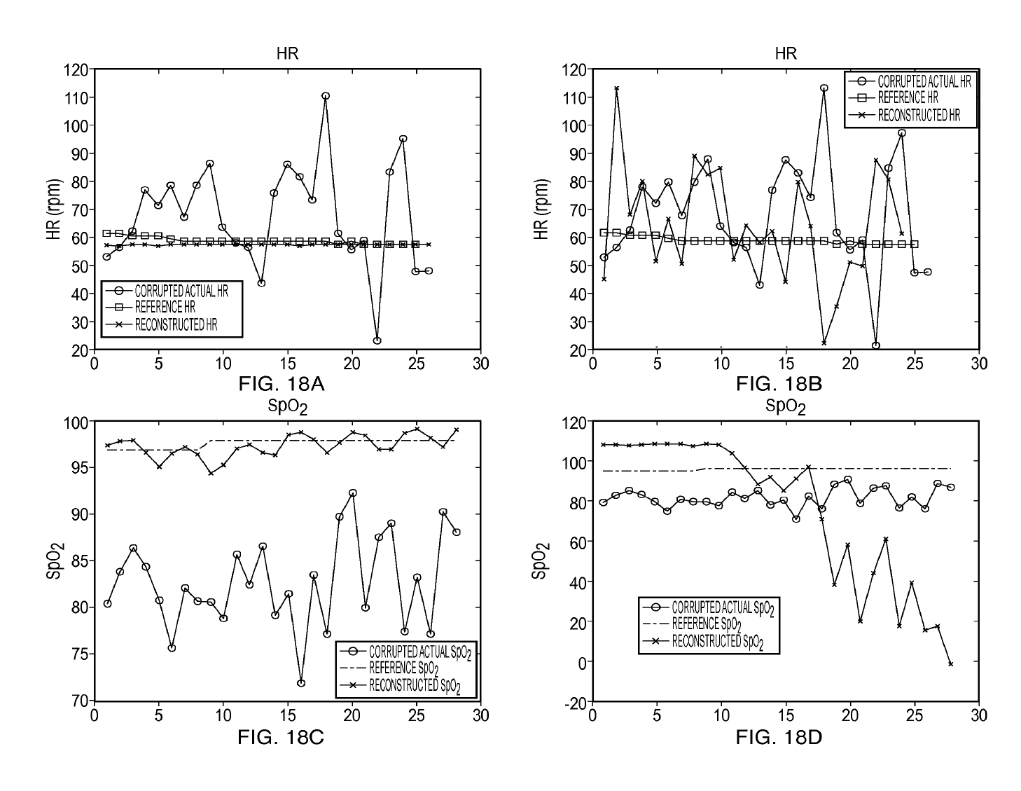

After applying the disclosed IMAR procedure to the identified MNA segment of the PPG signal, the SpO2 (using Eqs. 16-17) and HR are estimated and compared to the corresponding reference and MNA contaminated segment values. As is the case with the noise sensitivity analysis section, the performance of the IMAR algorithm is compared to the ICA method. The top panel (FIGS. 18A and 18B) and bottom panel (FIGS. 18C and 18D) of FIG. 18 represent a representative HR and SpO2 comparison result, respectively. These figures show that the estimated values for both HR (left panels) and SpO2 (right panels) from the IMAR (black font) track closely to the reference values recorded by the Masimo transmittance type finger pulse oximeter (red square line), while the estimated HR and SpO2 obtained from the ICA method (green font) deviate significantly from the reference signal. Below Tables X and XI show comparison of the IMAR and the ICA reconstructed HR and SpO2 values, respectively, for all 10 subjects. As shown in Table X, there is no significant difference between the finger reference HR and the IMAR reconstructed HR in 6 out of 10 subjects. However, there is significant difference between the finger reference HR and the ICA reconstructed HR in all 10 subjects. Similarly, the reconstructed SpO2 values from the IMAR are found to be not significantly different than the finger reference values in 6 out of subjects, but the ICA method is found to be significantly different for all 10 subjects.

TABLE X
Comparison & Statistical Analysis of HR Estimations from IMAR-
reconstructed PPG for 10 Different Subjects (Head Experiment).

| | | Finger HR | IMAR Recon- | ICA Recon- |
|---|---|---|---|---|
| Sub- | Head HR | (Reference) | structed HR | structed HR |
| ject | (mean _{±} std) | (mean _{±} std) | (mean _{±} std) | (mean _{±} std) |
| 1 | 68.31 _{±} 19.25 | 59.23 _{±} 1.49 | 59.76 _{±} 0.22* | 65.68 ± 20.98* |
| 2 | 85.39 _{±} 34.53 | 71.55 _{±} 3.037 | 73.72 _{±} 0.31* | 91.02 ± 35.48* |
| 3 | 76.19 _{±} 8.88 | 77.39 _{±} 1.360 | 78.705 _{±} 0.33 | 68.06 ± 14.14* |
| 4 | 94.47 _{±} 39.05 | 70.55 _{±} 3.686 | 73.66 _{±} 0.38* | 75.32 ± 13.42* |
| 5 | 72.33 _{±} 29.82 | 67.88 _{±} 4.643 | 66.83 _{±} 0.39 | 69.97 ± 20.20* |
| 6 | 45.09 _{±} 10.06 | 51.44 _{±} 1.481 | 49.00 _{±} 0.09* | 59.43 ± 22.97* |
| 7 | 44.82 _{±} 24.47 | 59.82 _{±} 1.486 | 57.56 _{±} 0.21 | 64.49 ± 35.63* |
| 8 | 63.46 _{±} 13.35 | 62.08 _{±} 0.865 | 62.23 _{±} 0.25 | 60.68 ± 10.70* |
| 9 | 59.37 _{±} 30.85 | 49.05 _{±} 1.555 | 49.19 _{±} 0.20 | 60.27 ± 13.24* |
| 10 | 46.89 _{±} 32.25 | 79.35 _{±} 1.323 | 78.93 _{±} 0.45 | 64.80 ± 25.60* |

*represents p < 0.05.

TABLE XI
Comparison & Statistical Analysis of SpO2 Estimations from IMAR-
reconstructed PPG for 10 Different Subjects (Head Experiment).

| | | Finger | IMAR Recon- | |
|---|---|---|---|---|
| | Head | SpO₂ | structed | ICA Recon- |
| Sub- | SpO₂ | (Reference) | SpO₂ | structed |
| ject | (mean _{±} std) | (mean _{±} std) | (mean _{±} std) | SpO₂ |
| 1 | 82.86 _{±} 4.86 | 97.70 _{±} 0.46 | 97.94 _{±} 0.93 | 76.721 ± 38.132* |
| 2 | 80.33 _{±} 2.82 | 97.67 _{±} 0.47 | 97.972 _{±} 4.048* | 111.097 ± 1.496* |
| 3 | 87.20 _{±} 4.54 | 95.41 _{±} 0.49 | 98.53 _{±} 0.727* | 74.081 ± 21.678* |
| 4 | 87.36 _{±} 2.64 | 97 _{±} 0 | 97.13 _{±} 0.23 | 81.391 ± 11.81* |
| 5 | 84.25 _{±} 3.76 | 98 _{±} 0 | 96.82 _{±} 5.25* | 77.593 ± 22.16* |
| 6 | 92.38 _{±} 2.64 | 98 _{±} 0 | 97.47 _{±} 0.97 | 84.069 ± 14.84* |
| 7 | 85.18 _{±} 3.06 | 98.41 _{±} 0.49 | 96.68 _{±} 0.38 | 75.632 ± 17.24* |
| 8 | 90.94 _{±} 2.38 | 99.82 _{±} 0.06 | 97.99 _{±} 0.38 | 89.322 ± 17.77* |
| 9 | 83.93 _{±} 4.54 | 98 _{±} 0 | 99.61 _{±} 3.87* | 100.15 ± 16.96* |
| 10 | 84.94 _{±} 4.24 | 95.97 _{±} 0.67 | 96.53 _{±} 4.62 | 86.731 ± 19.305* |

*represents p < 0.05.

Results—PPG Signal Reconstruction Performance in Finger Experiment

The performance of the signal reconstruction of the disclosed IMAR approach is compared to ICA for the PPG data with an index finger moving left-to-right patterns. The pulse oximeter on the middle finger of the right hand, which is stationary, is used as the reference signal. Since the subjects are directed to produce the motions for 30 seconds within each 1-minute segment, corresponding to 50% corruption by duration, the window length of both clean and corrupted segments are both set as half length of the signal. Table XII compares the HR reconstruction results between the IMAR and ICA methods for all 10 subjects. As shown in Table XII, the IMAR reconstructed HR values are not significantly different from the reference HR in 7 out of 10 subjects. However, the ICA's reconstructed HR is significantly different from the reference HR in 8 out of 10 subjects indicating poor reconstruction fidelity.

TABLE XII
Comparison & Statistical Analysis of HR Estimations
from IMAR-reconstructed PPG for 10 Different
Subjects (Finger Experiment).

| | | Finger HR | IMAR Recon- | ICA Recon- |
|---|---|---|---|---|
| Sub- | Head HR | (Reference) | structed HR | structed HR |
| ject | (mean _{±} std) | (mean _{±} std) | (mean _{±} std) | (mean _{±} std) |
| 1 | 77.43 ± 1.91 | 70.61 ± 0.73 | 70.42 ± 0.42 | 77.32 ± 8.34* |
| 2 | 63.60 ± 2.42 | 78.80 ± 0.41 | 78.36 ± 0.35 | 79.57 ± 9.68 |
| 3 | 70.82 ± 15.01 | 66.18 ± 0.76 | 67.21 ± 0.26 | 62.96 ± 22.53* |
| 4 | 87.70 ± 20.53 | 72.59 ± 0.26 | 70.85 ± 0.34 | 73.58 ± 11.34* |
| 5 | 84.34 ± 4.86 | 74.43 ± 0.29 | 73.51 ± 0.29* | 77.62 ± 18.55* |
| 6 | 81.75 ± 6.34 | 67.78 ± 0.36 | 69.07 ± 0.26* | 67.75 ± 18.01 |
| 7 | 63.75 ± 3.05 | 57.57 ± 0.54 | 58.32 ± 2.49 | 52.51 ± 24.06* |
| 8 | 66.75 ± 5.03 | 58.27 ± 0.75 | 60.34 ± 0.44* | 61.64 ± 28.83* |
| 9 | 97.27 ± 22.74 | 74.39 ± 0.46 | 74.25 ± 0.68 | 63.60 ± 14.96* |
| 10 | 73.76 ± 2.85 | 61.58 ± 0.50 | 61.40 ± 0.35 | 50.80 ± 13.72* |

*represents p < 0.05.

Results—PPG Signal Reconstruction Performance for the Walking and Stair Climbing Experimental Data

The signal reconstruction of the MNA identified data segments of the walking and stair climbing experiments using our disclosed IMAR and its comparison to ICA are provided in this section. Detection of the MNA data segments is performed using the algorithm described in Part I of the this disclosure. The reconstructed HR and SpO2 values using our disclosed algorithm and ICA are provided in below Tables XIII and XIV, respectively. For both HR and SpO2 reconstruction, the measurements are carried out using PPG data recorded from the head pulse oximeter. The right hand index finger's PPG data is used as HR and SpO2 references. As shown in Table XIII, 7 out of 9 subjects' reconstructed HR values are found to be not significantly different from the reference HR values using our algorithm. While 2 subjects' reconstructed HR values are found to be significantly different than the reference, the differences in the actual HR values are minimal. For ICA's reconstructed HR values, all values deviate significantly from the reference values.

TABLE XIII
Comparison & Statistical Analysis of HR Estimations from IMAR-reconstructed
PPG for 9 Different Subjects (Walking & Stair Climbing Experiment).

| | | Finger HR | IMAR Recon- | ICA Recon- |
|---|---|---|---|---|
| Sub- | Head HR | (Reference) | structed HR | structed HR |
| ject | (mean _{±} std) | (mean _{±} std) | (mean _{±} std) | (mean _{±} std) |
| 1 | 62.16 ± 18.96 | 70.73 ± 5.80 | 70.55 ± 0.56 | 77.39 ± 11.90* |
| 2 | 94.30 ± 20.37 | 94.40 ± 1.69 | 95.54 ± 0.86 | 92.94 ± 9.99* |
| 3 | 105.53 ± 17.23 | 120.64 ± 2.98 | 122.00 ± 1.05 | 95.67 ± 13.10* |
| 4 | 95.48 ± 8.37 | 101.61 ± 3.06 | 99.89 ± 0.44* | 90.89 ± 8.28* |
| 5 | 82.20 ± 13.07 | 86.99 ± 3.71 | 87.71 ± 1.07 | 82.84 ± 17.96* |
| 6 | 77.40 ± 12.69 | 82.48 ± 1.68 | 81.93 ± 0.48 | 86.81 ± 12.54* |
| 7 | 121.02 ± 19.26 | 107.58 ± 1.51 | 109.15 ± 0.07 | 138.62 ± 6.18* |
| 8 | 86.57 ± 9.85 | 91.95 ± 6.07 | 91.73 ± 0.57 | 80.44 ± 4.61* |
| 9 | 87.09 ± 6.56 | 82.55 ± 5.24 | 84.22 ± 1.93* | 104.30 ± 21.43* |

*represents p < 0.05.

TABLE XIV
Comparison & Statistical Analysis of SpO₂ Estimations from IMAR-reconstructed
PPG for 9 Different Subjects (Walking & Stair Climbing Experiment)

| | | Finger | IMAR Recon- | |
|---|---|---|---|---|
| | Head | SpO₂ | structed | ICA Recon- |
| Sub- | SpO₂ | (Reference) | SpO₂ | structed |
| ject | (mean _{±} std) | (mean _{±} std) | (mean _{±} std) | SpO₂ |
| 1 | 95.70 ± 7.62 | 99.00 ± 0 | 97.64 ± 2.50 | 84.21 ± 1.34* |
| 2 | 94.55 ± 5.51 | 95.37 ± 0 | 96.37 ± 0.99 | 95.53 ± 1.59 |
| 3 | 91.00 ± 15.58 | 96.75 ± 0 | 94.51 ± 0.42* | 84.64 ± 4.63* |
| 4 | 89.61 ± 3.36 | 99.62 ± 0 | 102.25 ± 0.65* | 87.33 ± 2.67* |
| 5 | 94.27 ± 8.12 | 98.00 ± 0.50 | 97.34 ± 1.45 | 76.50 ± 1.53* |
| 6 | 88.50 ± 13.95 | 96.00 ± 0.31 | 94.97 ± 4.07* | 82.94 ± 1.05* |
| 7 | 94.92 ± 16.77 | 98.00 ± 0 | 100.37 ± 3.15 | 90.69 ± 8.11* |
| 8 | 96.11 ± 6.60 | 97.00 ± 0 | 98.70 ± 4.16* | 96.11 ± 0.39 |
| 9 | 93.78 ± 6.60 | 98.62 ± 0 | 95.99 ± 2.39* | 89.11 ± 5.03* |

For the reconstructed SpO2 values, the disclosed algorithm again significantly outperforms ICA. All but one subject are not significantly different than the SpO2 reference values for ICA. For the disclosed IMAR algorithm, only 4 out of 9 subjects do not show significant difference from the reference values. Note the zero standard deviation reference SpO2 values from Massimo's pulse oximeter in 7 out of 9 subjects. This is because Massimo uses a proprietary averaging scheme based on several past values. Hence, it is possible that the significant difference seen with our algorithm in some of the subjects would turn out to be not significant if the averaging scheme are not used. While some of the SpO2 values from our algorithm are significantly different from the reference, the actual deviations are minimal and they are far less than with ICA.

### DISCUSSION

In this disclosure, a novel IMAR method is introduced to reconstruct MNA contaminated segments of PPG data. Detection of MNA using a support vector machine algorithm is introduced in the companion paper. One aim of this disclosure is to reconstruct the MNA corrupted segments as closely as possible to the non-corrupted data so that accurate heart rates and SpO2 values can be derived. The question is how to reconstruct the MNA data segments when there is no reference signal. To address this question, the most adjacent prior clean data segment and its dynamics are used to derive the MNA contaminated segment's heart rates and oxygen saturation values. Hence, the key assumption with the disclosed IMAR technique is that signal's dynamics do not change abruptly between the MNA contaminated segment and its most adjacent prior clean portion of data. Clearly, if this assumption is violated, the IMAR's ability to reconstruct the dynamics of the signal may be compromised. A time-varying IMAR algorithm can address this issue.

There are hosts of algorithms available for MNA elimination and signal reconstruction. Various adaptive filter approaches to remove MNA have been proposed with good results but the test data to fully evaluate the algorithms are either limited or confined to laboratory controlled MNA involving simple finger or arm movements. Moreover, these adaptive filter methods work best when a reference signal is available.

For those methods that do not require a reference signal to remove MNA, there have been many algorithms developed based on variants of the ICA. Most of the ICA-based methods produced reasonably good signal reconstructions of the MNA contaminated data. However, most of these methods are validated on data that are collected using laboratory controlled MNA involving pre-defined simple side-to-side or up-and-down finger and arm movements.

Given that ICA-based methods produced good signal reconstructions of the MNA contaminated data, the disclosed approach is compared to an ICA method using simulated data, laboratory controlled data as well as daily activity data involving both walking and stair climbing movements. Comparison of the performance of the disclosed method to ICA is based on reconstruction of HR and SpO2 values since these measures are currently used by clinicians.

Comparing HR and SpO2 estimations of the reconstructed signal to the reference measurements using both simulation and experimental data have shown that the proposed IMAR method is a promising tool as the reconstructed values are found to be accurate. The simulation results from noise sensitivity analysis showed that SNR level down to −20 dB and −dB from additive white and colored noise, respectively, can be tolerated well by the application of the proposed IMAR procedure, compared to the SNR values of −10 dB and −dB for the ICA method. Application of the proposed IMAR approach and the ICA to three different sets of experimental data have also shown significantly better signal reconstruction performance with our IMAR algorithm.

The use of singular spectrum analysis (SSA) to a single channel EEG recordings to extract high amplitude and low frequency MNA has been performed. The main aim of this work is to remove the artifacts in EEG signals, hence, an iterative approach to reconstruct the main dynamics of the signal is not implemented. The disclosed approach is based on the use of SSA combined with an iterative approach to reconstruct the portion of the MNA contaminated data with the most likely true dynamics (i.e., non-MNA contaminated data) of the pulse oximeter signal. This disclosure applies SSA-based algorithms for MNA reconstruction of pulse oximeter data. In conclusion, a scenario where a reference signal is not available to remove the MNA, the disclosed IMAR algorithm can accurately reconstruct HR and SpO2 values from MNA contaminated data segments.

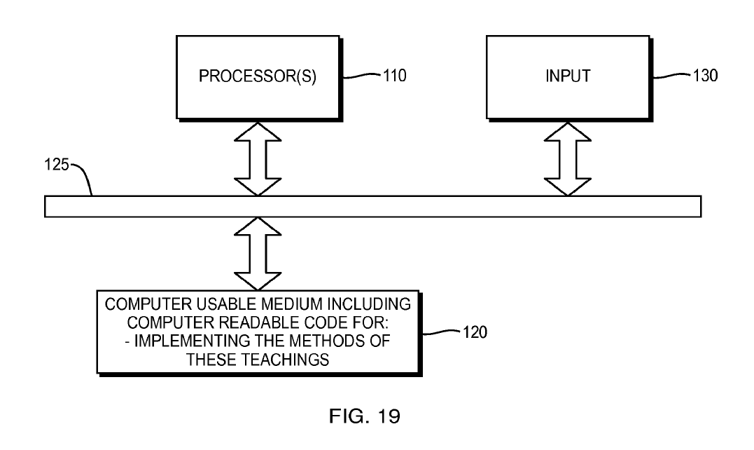

In one embodiment, the system of these teachings includes one or more processors and one or more computer usable media having computer readable code embodied therein, the computer readable code causing the one or more processors to execute the method of these teachings, shown in FIG. 19. Referring to FIG. 19, in the embodiment shown there in, one or more processors 110 are operatively connected to computer usable media 120 that has computer readable code embodied therein, which, when executed by the one or more processors 110, causes the one or more processors to perform the method of these teachings. An input device 130 is operatively connected to the one or more processors 110 and to the computer usable media 120 and enables the inputs of the PPG data segments. The one or more processors 110, the computer readable media 120 and the input device 130 are operatively connected by means of a computer connection component 125 (such as a computer bus).

In still another embodiment, the system of these teachings includes a system for determining whether MNA are present in a segment of PPG data, having one or more processors and non-transitory computer usable media having computer readable code embodied therein, the computer readable code, when executed by the one or more processors, causes the one or more processors to: determine a plurality of time domain features for each segment from a plurality of test segments of the PPG data, the plurality of test segments including segments without motion and noise artifacts and other segments with motion and noise artifacts, the plurality of time domain features for said each segment from the plurality of test segments constituting a training set; use the training set to train a SVM, training resulting in a trained SVM; determine the plurality of time domain features for the segment; and use the trained SVM to determine whether motion and noise artifacts are present in the segment. The computer readable code further causes the one or more processors to band pass filter, before determining the plurality of time domain features, each segment from the plurality of test segments. The computer readable code further causes the one or more processors to determine whether motion and noise artifacts are present in segments neighboring the segment, referred to as neighboring segments, neighboring segments being segments surrounding the segment within a predetermined time interval, and apply a majority vote algorithm to determinations of whether motion and noise artifacts are present in the segment and the neighboring segments. The time domain features comprise at least one of standard deviation of peak to peak interval within a segment, standard deviation of peak to peak amplitude within a segment, standard deviation of systolic and diastolic ratio within a segment, and mean standard deviation of pulse shape within an interval.

In yet another embodiment, the system of these teachings includes a system for removal of MNA present in a segment of PPG data, having one or more processors and non-transitory computer usable media, having computer readable code embodied therein, the computer readable code, when executed by the one or more processors, causes the one or more processors to: (a) for each one segment from a segment of PPG data in which presence of motion and noise artifacts has been previously detected, referred to as a corrupted segment, and a most prior adjacent segment of PPG data in which motion and noise artifacts are not detected, referred to as a clean segment, performing the following: (a1) assemble a data transition matrix, each row of the data transition matrix being a vector of a predetermined length, a number of vectors being equal to a number of samples in a segment for which the data transition matrix is assembled minus the predetermined length and plus one; a starting value of each vector being displaced by one sample from a previous vector, resulting in the data transition matrix having a number of columns equal to the predetermined length and a number of rows equal to the number of vectors; (a2) obtain eigenvectors and eigenvalues for the data transition matrix, resulting in eigenvectors and eigenvalues for the corrupted segment and eigenvectors and eigenvalues for the clean segment; (b) sorting the eigenvalues for the corrupted segment from largest to smallest; and sorting the eigenvalues for the clean segment from largest to smallest; (c) retaining only a top predetermined percentage of the eigenvalues for the corrupted segment and the eigenvalues for the clean segment; (d) replacing the eigenvalues for the corrupted segment with the eigenvalues for the clean segment, where only the top predetermined percentage of the eigenvalues and corresponding eigenvectors have been retained; (e) retaining only eigenvectors for the corrupted segment and eigenvectors for the clean segment that have data in a predetermined frequency range; (f) discarding eigenvectors for the corrupted segment that have different frequencies from the eigenvectors for the clean segment; (g) obtaining the data transition matrix for the corrupted segment from the eigenvalues and eigenvectors of the corrupted segment and the data transition matrix for the clean segment from the eigenvalues and eigenvectors of the clean segment; (h) repeating steps (a2) to (g) until a predetermined convergence criterion is satisfied; and (i) reconstructing, after the predetermined convergence criterion is satisfied, the corrupted segment from the data transition matrix for the corrupted segment using replaced eigenvalues and retained eigenvectors. The predetermined length is less than one half of a number of samples in the segment for which the data transition matrix is assembled and is larger than a ratio of a sampling frequency to a lowest frequency in said segment being considered. The predetermined convergence criterion comprises a difference between a discarding metric for the corrupted segment reconstructed from the data transition matrix using replaced eigenvalues and retained eigenvectors and a discarding metric for the clean segment; the discarding metric being a sum of absolute values of signal components divided by a length metric for the signal components. The predetermined frequency range is a heart rate range of PPG data. The predetermined frequency range includes frequencies greater than 0.66 Hz and less than 3 Hz. The top predetermined percentage is a top 5%. In this system, the presence of motion and noise artifacts has been previously detected using the system described above.

Elements and components described herein may be further divided into additional components or joined together to form fewer components for performing the same functions.

The following is a disclosure by way of example of a device configured to execute functions (hereinafter referred to as computing device) which may be used with the presently disclosed subject matter. The description of the various components of a computing device is not intended to represent any particular architecture or manner of interconnecting the components. Other systems that have fewer or more components may also be used with the disclosed subject matter. A communication device may constitute a form of a computing device and may at least include a computing device. The computing device may include an inter-connect (e.g., bus and system core logic), which can interconnect such components of a computing device to a data processing device, such as a processor(s) or microprocessor(s), or other form of partly or completely programmable or pre-programmed device, e.g., hard wired and or application specific integrated circuit (“ASIC”) customized logic circuitry, such as a controller or microcontroller, a digital signal processor, or any other form of device that can fetch instructions, operate on pre-loaded/pre-programmed instructions, and/or followed instructions found in hard-wired or customized circuitry to carry out logic operations that, together, perform steps of and whole processes and functionalities as described in the present disclosure.

Each computer program may be implemented in any programming language, such as assembly language, machine language, a high-level procedural programming language, or an object-oriented programming language. The programming language may be a compiled or interpreted programming language.

Each computer program may be implemented in a computer program product tangibly embodied in a computer-readable storage device for execution by a computer processor. Method steps of the invention may be performed by a computer processor executing a program tangibly embodied on a computer-readable medium to perform functions of the invention by operating on input and generating output.

In this description, various functions, functionalities and/or operations may be described as being performed by or caused by software program code to simplify description. However, those skilled in the art will recognize what is meant by such expressions is that the functions result from execution of the program code/instructions by a computing device as described above, e.g., including a processor, such as a microprocessor, microcontroller, logic circuit or the like. Alternatively, or in combination, the functions and operations can be implemented using special purpose circuitry, with or without software instructions, such as using Application-Specific Integrated Circuit (ASIC) or Field-Programmable Gate Array (FPGA), which may be programmable, partly programmable or hard wired. The application specific integrated circuit (“ASIC”) logic may be such as gate arrays or standard cells, or the like, implementing customized logic by metalization(s) interconnects of the base gate array ASIC architecture or selecting and providing metalization(s) interconnects between standard cell functional blocks included in a manufacturer's library of functional blocks, etc. Embodiments can thus be implemented using hardwired circuitry without program software code/instructions, or in combination with circuitry using programmed software code/instructions.

Thus, the techniques are limited neither to any specific combination of hardware circuitry and software, nor to any particular tangible source for the instructions executed by the data processor(s) within the computing device. While some embodiments can be implemented in fully functioning computers and computer systems, various embodiments are capable of being distributed as a computing device including, e.g., a variety of forms and capable of being applied regardless of the particular type of machine or tangible computer-readable media used to actually effect the performance of the functions and operations and/or the distribution of the performance of the functions, functionalities and/or operations.

The interconnect may connect the data processing device to define logic circuitry including memory. The interconnect may be internal to the data processing device, such as coupling a microprocessor to on-board cache memory or external (to the microprocessor) memory such as main memory, or a disk drive or external to the computing device, such as a remote memory, a disc farm or other mass storage device, etc. Commercially available microprocessors, one or more of which could be a computing device or part of a computing device, include a PA-RISC series microprocessor from Hewlett-Packard Company, an 80x86 or Pentium series microprocessor from Intel Corporation, a PowerPC microprocessor from IBM, a Sparc microprocessor from Sun Microsystems, Inc, or a 68xxx series microprocessor from Motorola Corporation as examples.

The inter-connect in addition to interconnecting such as microprocessor(s) and memory may also interconnect such elements to a display controller and display device, and/or to other peripheral devices such as input/output (I/O) devices, e.g., through an input/output controller(s). Typical I/O devices can include a mouse, a keyboard(s), a modem(s), a network interface(s), printers, scanners, video cameras and other devices which are well known in the art. The inter-connect may include one or more buses connected to one another through various bridges, controllers and/or adapters. In one embodiment the I/O controller includes a USB (Universal Serial Bus) adapter for controlling USB peripherals, and/or an IEEE-1394 bus adapter for controlling IEEE-1394 peripherals.

The memory may include any tangible computer-readable media, which may include but are not limited to recordable and non-recordable type media such as volatile and non-volatile memory devices, such as volatile RAM (Random Access Memory), typically implemented as dynamic RAM (DRAM) which requires power continually in order to refresh or maintain the data in the memory, and non-volatile ROM (Read Only Memory), and other types of non-volatile memory, such as a hard drive, flash memory, detachable memory stick, etc. Non-volatile memory typically may include a magnetic hard drive, a magnetic optical drive, or an optical drive (e.g., a DVD RAM, a CI) ROM, a DVD or a CD), or other type of memory system which maintains data even after power is removed from the system.

For the purposes of describing and defining the present teachings, it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Although these teachings have been described with respect to various embodiments, it should be realized these teachings are also capable of a wide variety of further and other embodiments within the spirit and scope of the appended claims.

1. A method for determining whether motion and noise artifacts (MNA) are present in a segment of photoplethysmography (PPG) data, the method comprising:
determining a plurality of time domain features for each segment from a plurality of test segments of the PPG data, the plurality of test segments including segments without motion and noise artifacts and other segments with motion and noise artifacts; the plurality of time domain features for said each segment from the plurality of test segments constituting a training set;
using the training set to train a support vector machine (SVM), training resulting in a trained SVM;
determining the plurality of time domain features for the segment; and
using the trained SVM to determine whether motion and noise artifacts are present in the segment.

2. The method of claim 1 further comprising:
band pass filtering, before determining the plurality of time domain features, each segment from the plurality of test segments.

3. The method of claim 1 further comprising:
determining whether motion and noise artifacts are present in segments neighboring the segment, referred to as neighboring segments; neighboring segments being segments surrounding the segment within a predetermined time interval; and
applying a majority vote algorithm to determinations of whether motion and noise artifacts are present in the segment and the neighboring segments.

4. The method of claim 1 wherein the time domain features comprise at least one of standard deviation of peak to peak interval within a segment, standard deviation of peak to peak amplitude within a segment, standard deviation of systolic and diastolic ratio within a segment, and mean standard deviation of pulse shape within an interval.

5. A method for removal of motion and noise artifacts (MNA) present in a segment of photoplethysmography (PPG) data, the method comprising:
(a) for each one segment from a segment of PPG data in which presence of motion and noise artifacts has been previously detected, referred to as a corrupted segment, and a most prior adjacent segment of PPG data in which motion and noise artifacts are not detected, referred to as a clean segment, performing the following:
(a1) assemble a data transition matrix, each row of the data transition matrix being a vector of a predetermined length, a number of vectors being equal to a number of samples in a segment for which the data transition matrix is assembled minus the predetermined length and plus one; a starting value of each vector being displaced by one sample from a previous vector, resulting in the data transition matrix having a number of columns equal to the predetermined length and a number of rows equal to the number of vectors;
(a2) obtain eigenvectors and eigenvalues for the data transition matrix, resulting in eigenvectors and eigenvalues for the corrupted segment and eigenvectors and eigenvalues for the clean segment;
(b) sorting the eigenvalues for the corrupted segment from largest to smallest; and sorting the eigenvalues for the clean segment from largest to smallest;
(c) retaining only a top predetermined percentage of the eigenvalues for the corrupted segment and the eigenvalues for the clean segment;
(d) replacing the eigenvalues for the corrupted segment with the eigenvalues for the clean segment, where only the top predetermined percentage of the eigenvalues and corresponding eigenvectors have been retained;
(e) retaining only eigenvectors for the corrupted segment and eigenvectors for the clean segment that have data in a predetermined frequency range;
(f) discarding eigenvectors for the corrupted segment that have different frequencies from the eigenvectors for the clean segment;
(g) obtaining the data transition matrix for the corrupted segment from the eigenvalues and eigenvectors of the corrupted segment and the data transition matrix for the clean segment from the eigenvalues and eigenvectors of the clean segment;
(h) repeating steps (a2) to (g) until a predetermined convergence criterion is satisfied; and
(i) reconstructing, after the predetermined convergence criterion is satisfied, the corrupted segment from the data transition matrix for the corrupted segment using replaced eigenvalues and retained eigenvectors.

6. The method of claim 5 wherein the predetermined length is less than one half of a number of samples in the segment for which the data transition matrix is assembled and is larger than a ratio of a sampling frequency to a lowest frequency in said segment being considered.

7. The method of claim 5 wherein the predetermined convergence criterion comprises a difference between a discarding metric for the corrupted segment reconstructed from the data transition matrix using replaced eigenvalues and retained eigenvectors and a discarding metric for the clean segment; the discarding metric being a sum of absolute values of signal components divided by a length metric for the signal components.

8. The method of claim 5 wherein the predetermined frequency range is a heart rate range of PPG data.

9. The method of claim 8 wherein the predetermined frequency range includes frequencies greater than 0.66 Hz and less than 3 Hz.

10. The method of claim 5 wherein the top predetermined percentage is a top 5%.

11. The method of claim 5 wherein the presence of motion and noise artifacts had been previously detected using the method of claim 1.

12. A system for determining whether motion and noise artifacts (MNA) are present in a segment of photoplethysmography (PPG) data, the system comprising:
one or more processors; and
non-transitory computer usable media, having computer readable code embodied therein, the computer readable code, when executed by the one or more processors, causes the one or more processors to:
determine a plurality of time domain features for each segment from a plurality of test segments of the PPG data, the plurality of test segments including segments without motion and noise artifacts and other segments with motion and noise artifacts; the plurality of time domain features for said each segment from the plurality of test segments constituting a training set;
use the training set to train a support vector machine (SVM), training resulting in a trained SVM;
determine the plurality of time domain features for the segment; and
use the trained SVM to determine whether motion and noise artifacts are present in the segment.

13. The system of claim 12 wherein the computer readable code further causes the one or more processors to:
band pass filter, before determining the plurality of time domain features, each segment from the plurality of test segments.

14. The system of claim 12 wherein the computer readable code further causes the one or more processors to:
determine whether motion and noise artifacts are present in segments neighboring the segment, referred to as neighboring segments; neighboring segments being segments surrounding the segment within a predetermined time interval; and
apply a majority vote algorithm to determinations of whether motion and noise artifacts are present in the segment and the neighboring segments.

15. The system of claim 12 wherein the time domain features comprise at least one of standard deviation of peak to peak interval within a segment, standard deviation of peak to peak amplitude within a segment, standard deviation of systolic and diastolic ratio within a segment, and mean standard deviation of pulse shape within an interval.

16. A system for removal of motion and noise artifacts (MNA) present in a segment of photoplethysmography (PPG) data, the system comprising:
one or more processors; and
non-transitory computer usable media, having computer readable code embodied therein, the computer readable code, when executed by the one or more processors, causes the one or more processors to:
(a) for each one segment from a segment of PPG data in which presence of motion and noise artifacts has been previously detected, referred to as a corrupted segment, and a most prior adjacent segment of PPG data in which motion and noise artifacts are not detected, referred to as a clean segment, perform the following:
(a1) assemble a data transition matrix, each row of the data transition matrix being a vector of a predetermined length, a number of vectors being equal to a number of samples in a segment for which the data transition matrix is assembled minus the predetermined length and plus one; a starting value of each vector being displaced by one sample from a previous vector, resulting in the data transition matrix having a number of columns equal to the predetermined length and a number of rows equal to the number of vectors;
(a2) obtain eigenvectors and eigenvalues for the data transition matrix, resulting in eigenvectors and eigenvalues for the corrupted segment and eigenvectors and eigenvalues for the clean segment;
(b) sort the eigenvalues for the corrupted segment from largest to smallest; and sort the eigenvalues for the clean segment from largest to smallest;
(c) retain only a top predetermined percentage of the eigenvalues for the corrupted segment and the eigenvalues for the clean segment;
(d) replace the eigenvalues for the corrupted segment with the eigenvalues for the clean segment, where only the top predetermined percentage of the eigenvalues and corresponding eigenvectors have been retained;
(e) retain only eigenvectors for the corrupted segment and eigenvectors for the clean segment that have data in a predetermined frequency range;
(f) discard eigenvectors for the corrupted segment that have different frequencies from the eigenvectors for the clean segment;
(g) obtain the data transition matrix for the corrupted segment from the eigenvalues and eigenvectors of the corrupted segment and the data transition matrix for the clean segment from the eigenvalues and eigenvectors of the clean segment;
(h) repeat steps (a2) to (g) until a predetermined convergence criterion is satisfied; and
(i) reconstruct, after the predetermined convergence criterion is satisfied, the corrupted segment from the data transition matrix for the corrupted segment using replaced eigenvalues and retained eigenvectors.

17. The system of claim 16 wherein the predetermined length is less than one half of a number of samples in the segment for which the data transition matrix is assembled and is larger than a ratio of a sampling frequency to a lowest frequency in said segment being considered.

18. The system of claim 16 wherein the predetermined convergence criterion comprises a difference between a discarding metric for the corrupted segment reconstructed from the data transition matrix using replaced eigenvalues and retained eigenvectors and a discarding metric for the clean segment; the discarding metric being a sum of absolute values of signal components divided by a length metric for the signal components.

19. The system of claim 16 wherein the predetermined frequency range is a heart rate range of PPG data.

20. The system of claim 19 wherein the predetermined frequency range includes frequencies greater than 0.66 Hz and less than 3 Hz.

21. The system of claim 16 wherein the top predetermined percentage is a top 5%.

22. The system of claim 16 wherein the presence of motion and noise artifacts had been previously detected using the system of claim 12.
